(19) United States
(12) Patent Application Publication
(10) Pub. No.: US 2026/0357358 A1
(43) Pub. Date: Nov. 12, 2026

(54) PLATFORM FOR CHARGE-DETECTION MASS SPECTROMETRY ANALYSIS OF AAVS

(71) Applicant: Regeneron Pharmaceuticals, Inc., Tarrytown, NY (US)

(72) Inventors: Chen DU, White Plains, NY (US); Shunhai WANG, Scarsdale, NY (US); Ning LI, New Canaan, CT (US)

(21) Appl. No.: 19/696,531

(22) Filed: Jun. 3, 2026

Related U.S. Application Data
(63) Continuation-in-part of application No. PCT/US2025/014183, filed on Jan. 31, 2025.
(60) Provisional application No. 63/548,636, filed on Feb. 1, 2024.

(51) Int. Cl. C12Q 1/70; C12Q 1/37; C12Q 1/68

(52) U.S. Cl. CPC C12Q 1/70; C12Q 1/37; C12Q 1/68; C12Y 301/21001; C12Y 304/21004

(57) ABSTRACT

The present invention provides systems and methods to identify, characterize and quantify viral vectors. Viral vector samples can be subjected to size exclusion chromatography-based desalting using a high flow and subsequently injected into an electrospray ion source using a low flow for charge detection mass spectrometry analysis. More particularly, the present disclosure provides a method of assessing an AAV sample that contains AAV capsids housing genetic material, by: subjecting the AAV sample to enzymatic digestion; and subjecting the digested sample to size exclusion chromatography (SEC) and/or charge-detection mass spectroscopy (CDMS) to detect AAV degradation products in the AAV sample.

### RELATED APPLICATIONS

The present application is a continuation-in-part of International Application No. PCT/US2025/014183 filed on Jan. 31, 2025, which claims benefit of U.S. Provisional Application Ser. No. 63/548,636, filed on Feb. 1, 2024, all of the contents of which are incorporated herein by reference.

### BACKGROUND

Gene therapy biopharmaceuticals mediate therapeutic effects by transcription and/or translation of transferred genetic material, such as integrating genetic material into the host genome, and are used to treat, prevent or cure a disease. Currently, gene therapy is one of the most investigated therapeutic modalities in preclinical and clinical settings. However, concerns have been raised about the safety of gene therapy and the critical need for safer gene delivery vectors. A better understanding of gene delivery vectors and advancing the manufacture of safe and effective vectors is necessary to mitigate safety risks.

Gene delivery vectors are important to ensure efficient gene delivery to the target tissue and cells. The ideal gene delivery system should have high gene transfer efficiency, low toxicity to the cell, and single cell specificity to the intended target. Based on gene delivery vector types, vectors can be divided into non-viral vectors and viral vectors. Due to the high gene transfer efficiency of viral vectors, they have been widely used in clinical trials.

Adeno-associated virus (AAV) is the most widely used viral vector for in vivo gene therapy applications. AAVs have low immunogenicity and they can enable long-term, stable gene expression. The use of AAVs for gene therapy has created the need for analytical methods to monitor and characterize these products. Process related and product related impurities should be monitored to ensure product quality and process consistency.

Biopharmaceutical products should meet very high standards of purity. Thus, it is important to monitor any impurities in such biopharmaceutical products at different stages of drug development, production, storage and handling. Residual impurities should be at an acceptable low level prior to conducting clinical studies. Residual impurities are also a concern for biopharmaceutical products intended for end-users. For example, an inherent characteristic of the AAV manufacturing process is the production of empty capsids, a capsid which lacks any packaged transgene. The presence of empty capsids in drug products should be monitored and can be unacceptable above a certain threshold. Sometimes, even empty capsids could result in an unwanted immune response and may reduce the potency of the gene therapy drug.

Charge-detection mass spectrometry (CDMS) can simultaneously measure charge and m/z of highly heterogeneous macromolecules and has emerged as an effective analytical method for the intact mass analysis of AAVs. CDMS is typically performed using static nano-electrospray ionization-based direct infusion with long acquisition times, which often suffers from unstable spray and sample instability.

It will be appreciated that a need exists for effective and reliable systems and methods to characterize viral vectors, such as characterizing capsid content in a drug substance or other product, to mitigate safety risks.

### SUMMARY

This disclosure sets forth newly developed size-exclusion chromatography (SEC)-based flow injection (FI) systems and methods to enable online desalting and long-lasting infusion for native MS and CDMS measurement of AAVs and AAV-antibody conjugates.

This disclosure provides systems for identifying, characterizing, and/or quantitating a polypeptide or viral vector of interest. In some exemplary embodiments, the systems can comprise a liquid chromatography system, comprising a first pump coupled to a size exclusion chromatography column, wherein said column is further coupled to a sample loop, and a second pump coupled to said sample loop; and a mass spectrometry system, comprising a microflow nanospray ion source, wherein said mass spectrometry system is coupled to said sample loop, and said mass spectrometry system is configured to perform charge detection mass spectrometry analysis of a polypeptide or viral vector.

In one aspect, said first pump is configured for a flow rate of about 100 μL/minute to about 300 μL/minute. In another aspect, said first pump is configured for a flow rate of about 200 μL/minute. In a further aspect, said first pump is configured for a flow rate of about 10 μL/minute to about 300 μL/minute. In yet another aspect, said first pump is configured for a flow rate of about 10 μL/minute, about 20 μL/minute, 50 μL/minute, about 75 μL/minute, about 100 μL/minute, about 125 μL/minute, about 150 μL/minute, about 175 μL/minute, about 200 μL/minute, about 225 μL/minute, about 250 μL/minute, about 275 μL/minute, or about 300 μL/minute. In yet another aspect, said first pump is configured for a flow rate of about 20 μL/minute. In yet another aspect, said first pump is configured for a flow rate of about 200 μL/minute.

In one aspect, said size exclusion chromatography column has an internal diameter of about 4 mm to about 5 mm. In another aspect, said size exclusion chromatography column has an internal diameter of about 4.6 mm. In a further aspect, said size exclusion chromatography column has an internal diameter of about 0.5 mm to about 1.5 mm, about 1 mm to about 2 mm, or about 1 mm to about 5 mm. In yet another aspect, said size exclusion chromatography column has an internal diameter of about 1 mm or about 1.5 mm. In yet another aspect, said size exclusion chromatography column has an internal diameter less than about 5 mm, about 4.5 mm, about 4 mm, about 3.5 mm, about 3 mm, about 2.5 mm, about 2 mm, about 1.5 mm, or about 1 mm.

In one aspect, said size exclusion chromatography column has a length of about 100 mm to about 200 mm. In another aspect, said size exclusion chromatography column has a length of about 150 mm. In a further aspect, said size exclusion chromatography column has a length of about 40 mm to about 60 mm or about 40 mm to about 200 mm. In yet another aspect, said size exclusion chromatography column has a length of about 50 mm, about 75 mm, about 100 mm, about 125 mm, about 150 mm, about 175 mm, or about 200 mm.

In one aspect, said sample loop has a volume of about 100 μL to about 300 μL. In another aspect, said sample loop has a volume of about 200 μL. In a further aspect, said sample loop has a volume of about 50 μL to about 100 μL, about 50 μL to about 300 μL, or about 100 μL to about 300 μL. In yet another aspect, said sample loop has a volume of about 60 μL, about 70 μL, about 80 μL, about 90 μL, about 100 μL, about 110 μL, about 120 μL, about 130 μL, about, 140 μL, about 150 μL, about 160 μL, about 170 μL, about 180 μL, about 190 μL, or about 200 μL. In yet another aspect, said sample loop has a volume of about 60 μL.

In one aspect, said second pump is configured for a flow rate of about 0.5 μL/minute to about 2 μL/minute. In another aspect, said second pump is configured for a flow rate of about 0.5 μL/minute, about 1 μL/minute, about 1.5 μL/minute, about 2 μL/minute, or about 2.5 μL/minute. In another aspect, said second pump is configured for a flow rate of about 2 μL/minute.

In one aspect, the system further comprises a gas input to the microflow nanospray ion source configured at a flow rate of about 0 μL/min to about 2 μL/min, about 0 L/min to about 1 μL/min, or about 1 μL/min to about 2 μL/min. In another aspect, the gas is a sheath gas.

In one aspect, said first pump is configured for a flow rate that is greater than the flow rate for which said second pump is configured.

In one aspect, said sample loop comprises tubing connecting an output of said size exclusion chromatography column and an input of said microflow nanospray ion source.

In one aspect, said polypeptide or viral vector of interest is an adeno-associated viral vector.

In one aspect, said adeno-associated viral vector comprises a serotype selected from the group consisting of AAV1, AAV2, AAV2quad(Y-F), AAV3, AAV4, AAV5, AAV6, AAV7, AAV8, AAV9, AAV10, AAV11, AAV12, AAV13 rh10, rh39, rh43, rh74, Avian AAV, Sea Lion AAV, Bearded Dragon AAV, and variations thereof, and combinations thereof.

This disclosure further provides methods for identifying, characterizing, and/or quantitating a polypeptide or viral vector of interest. In some exemplary embodiments, the methods can comprise (a) subjecting a sample including a polypeptide or viral vector of interest to size exclusion chromatography separation at a first flow rate to form a desalted sample; (b) injecting said desalted sample into an electrospray ion source at a second flow rate to form ions; and (c) subjecting said ions to charge detection mass spectrometry analysis to identify, characterize, and/or quantitate said polypeptide or viral vector of interest.

In one aspect, said polypeptide or viral vector of interest is an adeno-associated viral vector. In a specific aspect, said adeno-associated viral vector comprises a serotype selected from the group consisting of AAV1, AAV2, AAV2quad(Y-F), AAV3, AAV4, AAV5, AAV6, AAV7, AAV8, AAV9, AAV10, AAV11, AAV12, AAV13, rh10, rh39, rh43, rh74, Avian AAV, Sea Lion AAV, Bearded Dragon AAV, variations thereof, and combinations thereof.

In one aspect, said viral vector of interest is an antibody-viral vector conjugate. In a specific aspect, said characterizing comprises quantitating a number of antibodies in said antibody-viral vector conjugate. In another aspect, the viral vector can comprise a first cognate member of a specific binding pair covalently bound together, and the antibody is linked to a second cognate member of the specific binding pair covalently bound together. The first member and second cognate member can be a system selected from the group consisting of Spy Tag:Spy Catcher, Spy Tag002:SpyCatcher002, SpyTag003:SpyCatcher003, SpyTag:KTag, SnoopTag:SnoopCatcher, Isopeptag:Pilin-C, Isopeptag:Pilin-N, SnoopTagJr:SnoopCatcher, DogTag:DogCatcher, SdyTg:SdyCatcher, Jo:In, 3kptTag: 3kptCatcher, 4oq1Taq/4oq1Catcher, NGTag/NGCatcher, Rumtrunk/Mooncake, Snoop ligase, GalacTag, Cpe, Ececo, and Corio. Spy Tag002:SpyCatcher002 and SpyTag003:SpyCatcher003 are different iterations of Spy Tag:Spy Catcher.

In one aspect, said size exclusion chromatography separation and said mass spectrometry analysis are performed under native conditions.

In one aspect, a mobile phase for said size exclusion chromatography is ammonium acetate. In a specific aspect, a concentration of said ammonium acetate is about 100 mM to about 200 mM. In another specific aspect, a concentration of said ammonium acetate is about 150 mM.

In one aspect, said first flow rate is about 100 μL/minute to about 300 μL/minute. In another aspect, said first flow rate is about 200 μL/minute. In a further aspect, said first pump is configured for a flow rate of about 10 μL/minute to about 300 μL/minute. In yet another aspect, said first pump is configured for a flow rate of about 10 μL/minute, about 20 μL/minute, 50 μL/minute, about 75 μL/minute, about 100 μL/minute, about 125 μL/minute, about 150 μL/minute, about 175 μL/minute, about 200 μL/minute, about 225 μL/minute, about 250 μL/minute, about 275 μL/minute, or about 300 μL/minute. In yet another aspect, said first pump is configured for a flow rate of about 20 μL/minute. In yet another aspect, said first pump is configured for a flow rate of about 200 μL/minute.

In one aspect, said first flow rate is controlled by a first pump.

In one aspect, said desalted sample is contained in a sample loop. In a specific aspect, said sample loop comprises tubing connecting said size exclusion chromatography column and said electrospray ion source. In another specific aspect, a volume of said sample loop is about 100 μL to about 300 μL. In a further specific aspect, a volume of said sample loop is about 200 μL. In a further aspect, said sample loop has a volume of about 50 μL to about 100 μL, about 50 μL to about 300 μL, or about 100 μL to about 300 μL. In yet another aspect, said sample loop has a volume of about 60 μL, about 70 μL, about 80 μL, about 90 μL, about 100 μL, about 110 μL, about 120 μL, about 130 μL, about, 140 μL, about 150 μL, about 160 μL, about 170 μL, about 180 μL, about 190 μL, or about 200 μL. In yet another aspect, said sample loop has a volume of about 60 μL.

In one aspect, said second flow rate is about 0.5 μL/minute to about 2 μL/minute. In another aspect, said second pump is configured for a flow rate of about 0.5 μL/minute, about 1 μL/minute, about 1.5 μL/minute, about 2 μL/minute, or about 2.5 μL/minute. In another aspect, said second pump is configured for a flow rate of about 2 μL/minute.

In one aspect, said second flow rate is controlled by a second pump. In another aspect, said first flow rate is greater than said second flow rate.

In one aspect, said electrospray ion source is a microflow nanospray ion source.

In one aspect, said injecting is performed for from 30 minutes to 240 minutes. In another aspect, said injecting is performed for about 30 minutes, about 60 minutes, about 180 minutes, or about 240 minutes.

In one aspect, the method further comprises subjecting the results of said charge detection mass spectrometry analysis to frequency correction.

In one aspect, the method further comprises injecting said desalted sample into said electrospray ion source at said first flow rate to form ions, and subjecting said ions to mass spectrometry analysis to further identify, characterize, and/or quantitate said polypeptide or viral vector of interest.

In another aspect, the present disclosure is directed to charge detection mass spectrometry (CDMS) and native digestion strategies to elucidate AAV degradation pathways under extended thermal stress. Using AAV1 and AAV8 as model systems, it is herein demonstrated that genome release proceeds through intermediate DNA-extrusion states that retain the mass of full capsids while exhibiting elevated charge states. DNA ejection ultimately drives the conversion of full capsids into empty forms, generating distinct, structurally compromised intermediates influenced by serotype, genome size, and the degree of thermal stress. Furthermore, DNA extrusion promotes cooperative externalization of the VP1u and VP1/2 regions, facilitating DNA-viral protein-mediated aggregation that can be largely reversed by native enzymatic digestion of either the DNA or protein components. Together, these findings reveal previously uncharacterized AAV degradation mechanisms and establish an effective analytical framework for AAV stability assessment.

More particularly, the present disclosure is directed to a method of assessing an AAV sample that contains AAV capsids housing genetic material, the method comprising subjecting the AAV sample to enzymatic digestion; and subjecting the digested sample to size exclusion chromatography (SEC) and/or charge-detection mass spectroscopy (CDMS) to detect AAV degradation products in the AAV sample.

In some embodiments, the enzymatic digestion is achieved by contacting the AAV sample with DNAse or protease (e.g., trypsin).

In some embodiments, the degradation products comprise empty capsids, capsids with partially externalized DNA, and/or aggregated AAV species.

In some embodiments, the empty capsids lack one or more VP regions.

In some embodiments, the method further comprises identifying and/or quantifying one or more degradation products in the AAV sample.

In some embodiments, the method further comprises quantifying the ratio of empty capsids to full capsids.

In some embodiments, the method further comprises quantifying empty capsids missing one or more VP regions.

In some embodiments, the method further comprises quantifying capsids with partially externalized DNA.

In some embodiments, the enzymatic digestion converts aggregated AAV species into monomeric AAV species, and wherein the method further comprises quantifying the amount of aggregated AAV species in the AAV sample by comparing SEC/CDMS results of the enzyme-digested AAV sample with SEC/CDMS results of the undigested AAV sample.

In some embodiments, the AAV sample, before enzyme digestion, has been subjected to a condition that induces degradation of AAV. In some embodiments, the condition that induces degradation is a temperature above room temperature.

In some embodiments, the SEC is SEC-FLR/UV, which is SEC equipped with fluorescence and ultraviolet dual detection.

In some embodiments, the CDMS contains an electrospray ion source (EIS). In some embodiments, the EIS is a microflow nanospray ion source.

In some embodiments, the SEC is integrated with the CDMS by direct flow injection of mobile phase from the SEC into the CDMS, wherein the mobile phase is subjected to a desalting mechanism before being injected into the CDMS. In some embodiments, the desalting mechanism is achieved using a high-flow configuration that provides online desalting and native mass spectroscopy (MS) screening, wherein said high-flow configuration employs a flow rate of about 10 μL/min to about 30 μL/min through the SEC column to result in a desalted sample. In some embodiments, the desalted sample is injected into an EIS of the CDMS. In further embodiments, a sample loop contains the desalted sample and connects the SEC with the EIS. In some embodiments, the desalted sample is injected into the EIS using a low-flow configuration that employs a flow rate of about 0.5 μL/min to about 2 μL/min.

In some embodiments, the SEC employs a mobile phase that contains a volatile buffer. In some embodiments, the volatile buffer is ammonium acetate.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a schematic diagram of an Orbitrap analyzer for charge-detection mass spectrometry (CDMS) analysis, according to an exemplary aspect.

FIG. 1B depicts a calculation of ion mass using CDMS analysis, according to an exemplary aspect.

FIG. 1C depicts a comparison of a protein mass spectrum to a single ion CDMS, according to an exemplary aspect.

FIG. 1D depicts an example of highly glycosylated spike proteins that can be resolved using CDMS, according to an exemplary aspect.

FIG. 1E depicts an example of large AAV vectors that can be resolved using CDMS, according to an exemplary aspect.

FIG. 2A depicts single ion data acquisition for STORI analysis of Orbitrap-based CDMS, according to an exemplary aspect.

FIG. 2B depicts analysis of ion data using STORI analysis, according to an exemplary aspect.

FIG. 2C depicts data processing of STORI analysis, according to an exemplary aspect.

FIG. 3A depicts a static infusion system for an ultra-high mass range (UHMR) mass spectrometry system, according to an exemplary aspect.

FIG. 3B depicts a total ion chromatogram (TIC) trace of static infusion of an AAV sample, according to an exemplary aspect.

FIG. 4A depicts a high flow configuration of a size exclusion chromatography-flow injection (SEC-FI) MS system, according to an exemplary aspect.

FIG. 4B depicts a low flow configuration of a SEC-FI-CDMS system, according to an exemplary aspect.

FIG. 5A depicts efficient online desalting of an AAV8 sample using high-flow SEC-FI (native MS), according to an exemplary aspect.

FIG. 5B depicts efficient stable infusion of an AAV8 sample using low-flow SEC-FI-CDMS (single ion CMDS), according to an exemplary aspect.

FIG. 6A depicts optimized parameters for SEC-FI-CDMS, according to an exemplary aspect.

FIG. 6B depicts an optimized low flow configuration SEC-FI-CDMS system, according to an exemplary aspect.

FIG. 6C depicts a comparison of a non-optimized (“Standard”) SEC-FI-MS analysis and an optimized (“AAV”) SEC-FI-MS analysis, according to an exemplary aspect.

FIG. 6D depicts a comparison of a non-optimized (“Standard”) SEC-FI-CDMS analysis and an optimized (“AAV”) SEC-FI-CDMS analysis, according to an exemplary aspect.

FIG. 7A depicts mass measurements of empty and full AAV8 using Orbitrap-based SEC-FI-CDMS, according to an exemplary aspect.

FIG. 7B depicts a comparison of SEC-FI-CDMS analysis with and without sheath gas, according to an exemplary aspect.

FIG. 8A depicts a comparison of SEC-FI-MS analysis of empty AAV8 with and without STORI frequency correction, according to an exemplary aspect.

FIG. 8B depicts a comparison of SEC-FI-MS analysis of full AAV8 with and without STORI frequency correction, according to an exemplary aspect.

FIG. 9A depicts native MS analysis of VP3-only AAV using static infusion, according to an exemplary aspect.

FIG. 9B depicts SEC-FI-CDMS analysis of VP3-only AAV, according to an exemplary aspect.

FIG. 10A depicts SEC-FI-CDMS analysis of VP3-only AAV at varying mixing ratios, according to an exemplary aspect.

FIG. 10B depicts a comparison of calculated and measured VP3-only AAV at varying mixing ratios, according to an exemplary aspect.

FIG. 11A depicts total ion chromatograms from high-flow SEC-FI analysis of a sample of mixed empty and full AAV vectors, according to an exemplary aspect.

FIG. 11B depicts SEC-FI-CDMS analysis of full and empty AAV at varying mixing ratios, according to an exemplary aspect.

FIG. 11C depicts a comparison of predicted and measured masses of full and empty AAV, according to an exemplary aspect.

FIG. 11D depicts a comparison of SEC-FI-CDMS measurements of full and empty AAV masses across replicates, according to an exemplary aspect.

FIG. 12A depicts native SEC-FI-MS analysis of full and empty AAV at varying mixing ratios, according to an exemplary aspect.

FIG. 12B depicts SEC-FI-CDMS analysis of full and empty AAV at varying mixing ratios, according to an exemplary aspect.

FIG. 12C depicts a comparison of calculated and measured full AAV at varying mixing ratios, according to an exemplary aspect.

FIG. 12D depicts a comparison of full AAV measured at varying mixing ratios using AUC or native SEC-FI-MS, according to an exemplary aspect.

FIG. 12E depicts a comparison of full AAV measured at varying mixing ratios using AUC or SEC-FI-CDMS, according to an exemplary aspect.

FIG. 13A depicts native SEC-FI-MS analysis of empty AAV8 capsid at varying injection amounts, according to an exemplary aspect.

FIG. 13B depicts SEC-FI-CDMS analysis of empty AAV8 capsid at varying injection amounts, according to an exemplary aspect.

FIG. 14A depicts a measurement of empty, partially filled, and full AAV vectors using low-flow SEC-FI analysis for a first AAV8 lot, according to an exemplary aspect.

FIG. 14B depicts a measurement of empty, partially filled, and full AAV vectors using low-flow SEC-FI analysis for a second AAV8 lot, according to an exemplary aspect.

FIG. 14C depicts a measurement of empty, partially filled, and full AAV vectors using low-flow SEC-FI analysis for a third AAV8 lot, according to an exemplary aspect.

FIG. 14D depicts a measurement of empty, partially filled, and full AAV vectors using low-flow SEC-FI analysis for a fourth AAV8 lot, according to an exemplary aspect.

FIG. 14E depicts a comparison of measurements of empty, partially filled, and full AAV vectors using low-flow SEC-FI analysis across four AAV8 lots, according to an exemplary aspect.

FIG. 15A depicts a measurement of empty, partially filled, and full AAV1 and AAV5 vectors using SEC-FI-CDMS analysis, according to an exemplary aspect.

FIG. 15B depicts a comparison of expected and measured empty, partially filled, and full AAV1 and AAV5 vectors, according to an exemplary aspect.

FIG. 15C depicts a comparison of measured empty, partially filled, and full AAV1 and AAV5 vectors using AUC or SEC-FI-CDMS, according to an exemplary aspect.

FIG. 16A illustrates an antibody-AAV conjugate, according to an exemplary aspect.

FIG. 16B illustrates a legend for FIGS. 16C-16H, according to an exemplary aspect.

FIG. 16C depicts a mass spectrum of a sample with a 1:0 ratio of AAV9-SpyT:SpyC-mAb obtained using low-flow SEC-FI-CDMS, according to an exemplary aspect.

FIG. 16D depicts a mass spectrum of a sample with a 1:4 ratio of AAV9-SpyT:SpyC-mAb obtained using low-flow SEC-FI-CDMS, according to an exemplary aspect.

FIG. 16E depicts a mass spectrum of a sample with a 1:8 ratio of AAV9-SpyT:SpyC-mAb obtained using low-flow SEC-FI-CDMS, according to an exemplary aspect.

FIG. 16F depicts a mass spectrum of a sample with a 1:12 ratio of AAV9-SpyT:SpyC-mAb obtained using low-flow SEC-FI-CDMS, according to an exemplary aspect.

FIG. 16G depicts a mass spectrum of a sample with a 1:16 ratio of AAV9-SpyT:SpyC-mAb obtained using low-flow SEC-FI-CDMS, according to an exemplary aspect.

FIG. 16H depicts a mass spectrum of a sample with a 1:100 ratio of AAV9-SpyT:SpyC-mAb obtained using low-flow SEC-FI-CDMS, according to an exemplary aspect.

FIG. 17A depicts measured masses of AAV-antibody conjugates using SEC-FI-CDMS, according to an exemplary aspect.

FIG. 17B depicts a relative abundance of AAV-antibody conjugate species, according to an exemplary aspect.

FIG. 18A depicts a SEC-FI-CDMS analysis of AAV surface charge as a function of anti-AAV antibody binding at a ratio of 1:0 of AAV9-SpyT:SpyC-mAb, according to an exemplary aspect.

FIG. 18B depicts a SEC-FI-CDMS analysis of AAV surface charge as a function of anti-AAV antibody binding at a ratio of 1:4 of AAV9-SpyT:SpyC-mAb, according to an exemplary aspect.

FIG. 18C depicts a SEC-FI-CDMS analysis of AAV surface charge as a function of anti-AAV antibody binding at a ratio of 1:8 of AAV9-SpyT:SpyC-mAb, according to an exemplary aspect.

FIG. 18D depicts a SEC-FI-CDMS analysis of AAV surface charge as a function of anti-AAV antibody binding at a ratio of 1:12 of AAV9-SpyT:SpyC-mAb, according to an exemplary aspect.

FIG. 18E depicts a SEC-FI-CDMS analysis of AAV surface charge as a function of anti-AAV antibody binding at a ratio of 1:16 of AAV9-SpyT:SpyC-mAb, according to an exemplary aspect.

FIG. 18F depicts a SEC-FI-CDMS analysis of AAV surface charge as a function of anti-AAV antibody binding at a ratio of 1:100 of AAV9-SpyT:SpyC-mAb, according to an exemplary aspect.

FIG. 19A depicts TIC traces from SEC-FI-CDMS charge calibration using various protein standards, according to an exemplary aspect.

FIG. 19B depicts charge calibration coefficients across four protein standards using the SEC-FI-CDMS platform, according to an exemplary aspect.

FIG. 20A depicts a measurement of empty, partially full, and full AAV1 vectors comprising a 3′ template (top) or a 5′ template (bottom) using SEC-FI-CDMS analysis, according to an exemplary aspect.

FIG. 20B depicts a comparison of measured empty, partially filled, and full AAV1 vectors comprising a 3′ template or a 5′ template, according to an exemplary aspect.

FIG. 21A depicts SEC-FI-CDMS analysis of full and empty AAV1 comprising a 3′ template and AAV1 comprising a 5′ template at varying mixing ratios, according to an exemplary aspect.

FIG. 21B depicts a comparison of measured full AAV1 comprising a 3′ template and AAV1 comprising a 5′ template at varying mixing ratios, according to an exemplary aspect.

FIG. 22A depicts SEC-CDMS analysis of empty and full AAV1 comprising a 3′ template or a 5′ template, according to an exemplary aspect.

FIG. 22B depicts SEC-CDMS analysis of empty and full AAV1 comprising a 3′ template or a 5′ template, according to an exemplary aspect.

FIG. 22C depicts SEC-CDMS analysis of empty and full AAV1 comprising a 3′ template or a 5′ template, according to an exemplary aspect.

FIG. 22D depicts SEC-CDMS analysis of empty and full AAV1 comprising a 3′ template or a 5′ template, according to an exemplary aspect.

FIG. 22E depicts SEC-CDMS analysis of empty and full AAV1 comprising a 3′ template or a 5′ template, according to an exemplary aspect.

FIG. 23A depicts a comparison of measured empty, partially filled, and full AAV1 comprising a 3′ template, a 5′ template, or a sample comprising both an AAV1-3′ template and an AAV1-5′ template at either time 0 (TO) or after incubated at 37 °C for 14 days (37 °C 14d), according to an exemplary aspect.

FIG. 23B depicts a comparison of the percentage of empty, partially filled, and full AAV1 based on the conditions as shown in FIG. 23A.

FIG. 24A depicts a comparison of measured empty, partially filled, full, and heavy molecular weight (HMW) species of AAV1 comprising a 3′ template, a 5′ template, or a sample comprising both an AAV1-3′ template and an AAV1-5′ template at either time 0 (TO) or after incubated at 37 °C for 14 days (37 °C 14d), according to an exemplary aspect.

FIG. 24B depicts SEC-CDMS analysis of empty, partially filled, full, and heavy molecular weight (HMW) species of AAV1 comprising a 3′ template, a 5′ template, or a sample comprising both an AAV1-3′ template and an AAV1-5′ template at either time 0 (TO) or after incubated at 37 °C for 14 days (37 °C 14d), according to an exemplary aspect.

FIG. 25 depicts mass v. charge heat maps of empty, partially filled, full, and heavy molecular weight (HMW) species of AAV1 comprising a 3′ template, a 5′ template, or a sample comprising both an AAV1-3′ template and an AAV1-5′ template before (e.g., time 0 (TO)) and after thermally stressed (e.g., 37 °C for 14 days (37 °C 14d)), according to an exemplary aspect.

FIG. 26A depicts comparison of measurement of AAV1-3′ template before and after thermal stress, according to an exemplary aspect.

FIG. 26B depicts high molecular weight (HMW) and low molecular weight (LMW) species of AAV1 3′ template after thermally stressed, according to an exemplary aspect.

FIG. 26C depicts mass v. charge heat map of empty, partially filled, full, and heavy molecular weight (HMW) and low molecular weight (LMW) species of AAV1 comprising a 3′ template, according to an exemplary aspect.

FIGS. 27A-27L. AAV1 capsid degradation under thermal stress studied by SEC-FLR/UV, CDMS, and native digestion strategies. FIGS. 27A-27C show SEC-FLR/UV profile, CDMS spectra, and charge-mass heatmap of AAV1 at TO; FIGS. 27D-27F show SEC-FLR/UV profile (zoom-in view at retention time 9-13 min), CDMS spectra, and charge-mass heatmap of stressed AAV1 at 37 °C for 14 days; FIGS. 27G-27I show SEC-FLR/UV profile (zoom-in view at retention time 9-13 min), CDMS spectra, and charge-mass heatmap of stressed AAV1 with native DNase treatment; and FIGS. 27J-27L show SEC-FLR/UV profile (zoom-in view at retention time 9-13 min), CDMS spectra, and charge-mass heatmap of stressed AAV1 with native trypsin treatment.

FIGS. 28A-28L. AAV8 capsid degradation under thermal stress studied by SEC-FLR/UV, CDMS, and native digestion strategies. FIGS. 28A-28C show SEC-FLR/UV profile, CDMS spectra, and charge-mass heatmap of AAV8 at TO; FIGS. 28D-28F show SEC-FLR/UV profile (zoom-in view at retention time 9-13 min), CDMS spectra, and charge-mass heatmap of stressed AAV8 at 45 °C for 28 days; FIGS. 28G-28I show SEC-FLR/UV profile (zoom-in view at retention time 9-13 min), CDMS spectra, and charge-mass heatmap of stressed AAV8 with native DNase treatment; and FIGS. 28J-28L show SEC-FLR/UV profile (zoom-in view at retention time 9-13 min), CDMS spectra, and charge-mass heatmap of stressed AAV8 with native trypsin treatment.

FIG. 29 (left and right). Overall DNA recovery and degree of DNA externalization of AAV1 (left graph) and AAV8 (right graph) samples at T0 and following thermal stress. Grey bars (native digestion) and blue bars (denatured digestion) show recovered DNA quantified by dT signal, normalized to the T0, denatured digests. The red dashed line indicates the degree of DNA externalization, obtained using the dT signal from native digestion normalized against the value by denatured digestion. Background dT signal from reagent control was subtracted to minimize the interference from environmental DNA.

FIG. 30 (top and bottom). Assessing the susceptibility of VP1 and VP2 to native trypsin digestion in AAV8 (top) and AAV1 (bottom) by dSEC-FLR/MS-based intact viral protein analysis. Thermally stressed (red, dashed traces) and unstressed (blue, solid traces) AAV samples were subjected to native trypsin digestion for different durations (t=0 h, 0.5 h, 4 h, and overnight). For each time point, the relative abundances of VP1 and VP2 (by FLR) were normalized to the corresponding values at to. Samples were prepared and analyzed in triplicates. A 0.5 h offset in x-axis was applied to VP2 data points to improve visualization.

FIG. 31. Scheme showing proposed AAV capsid degradation pathways under thermal stress and the interplay between DNA ejection and viral protein externalization.

### DETAILED DESCRIPTION

Adeno-associated viruses (AAVs) have been widely used as gene delivery vectors to deliver genetic material, such as delivering nucleic acids for gene therapy. AAVs provide the advantages of non-pathogenicity and low immunogenicity. AAVs are nonpathogenic members of the Parvoviridae family under Dependovirus genus and require helpers, such as Adenovirus or Herpesvirus, for infection (Venkatakrishnan et al., Structure and Dynamics of Adeno-Associated Virus Serotype 1 VP1—Unique N-Terminal Domain and Its Role in Capsid Trafficking, Journal of Virology, May, 2013, vol. 87, no. 9, pages 4974-4984). AAV encapsulates a single-stranded DNA genome of about 4.7 kilobases (kb) in an icosahedral capsid which is made of a shell of capsid viral proteins. Recombinant AAV genomes are nonpathogenic and do not integrate into a host's genome, but exist as stable episomes that provide long-term expression. AAV serotypes make a very useful system for preferentially transducing specific cell types.

Overall, AAV-based therapy has the advantages of being non-pathogenic and non-toxic, having cell type-specific infection, and offering different serotypes with varying cell transduction efficiencies. A disadvantage is that AAV production, purification, and characterization are more complex compared to, for example, antibody therapies. The purity of AAVs is defined by several product-related impurities, including empty capsids, capsids containing partial or incorrect genomes, and aggregated or degraded capsid, as well as residual host cell proteins (HCP).

A characteristic of the AAV manufacturing process is the production of empty capsids, namely, capsids lacking any packaged transgene. Empty capsids are impurities that may result in an unwanted immune response and may reduce potency of the gene therapy drug. An empty capsid has a molecular weight of about 3750 kDa, while a full capsid with an about 4.7 kb single-stranded genome has a molecular weight of about 5100 kDa. In addition to empty capsids, a heterogeneous population of partially filled capsids, containing process-related impurities or a truncated transgene, may also be produced during manufacturing. These impurities can lower the overall yield and therapeutic potential of the gene therapy product (full capsids). Aggregated or degraded capsids, which may present as low or high molecular weight species in a sample, also must be monitored, as they may reduce viral titer and efficacy while increasing the load of potentially immunogenic viral protein. The ratio of empty-full capsids, empty-partially filled-full capsids, the presence of degraded or aggregated capsids, and the presence of low molecular weight species or high molecular weight species may be critical quality attributes (CQA) for a viral vector gene therapy product.

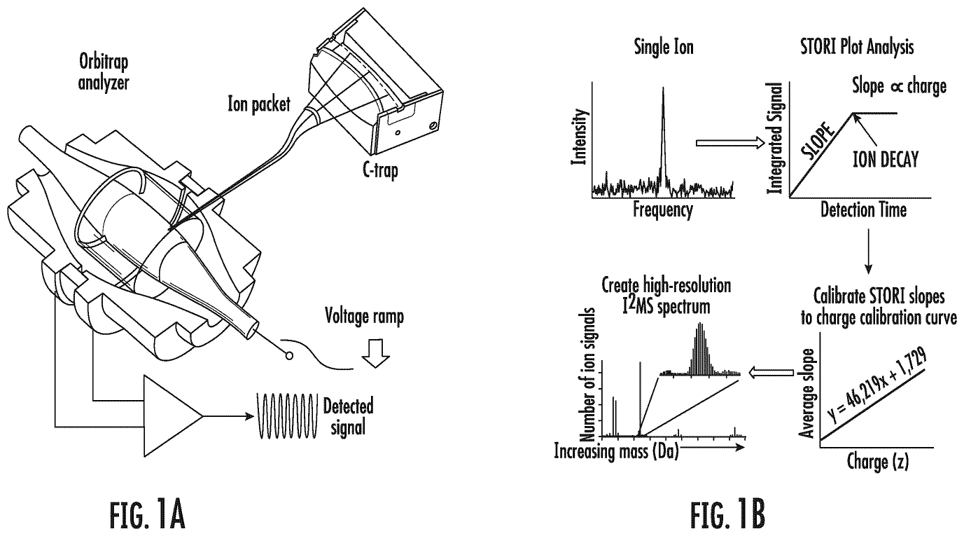

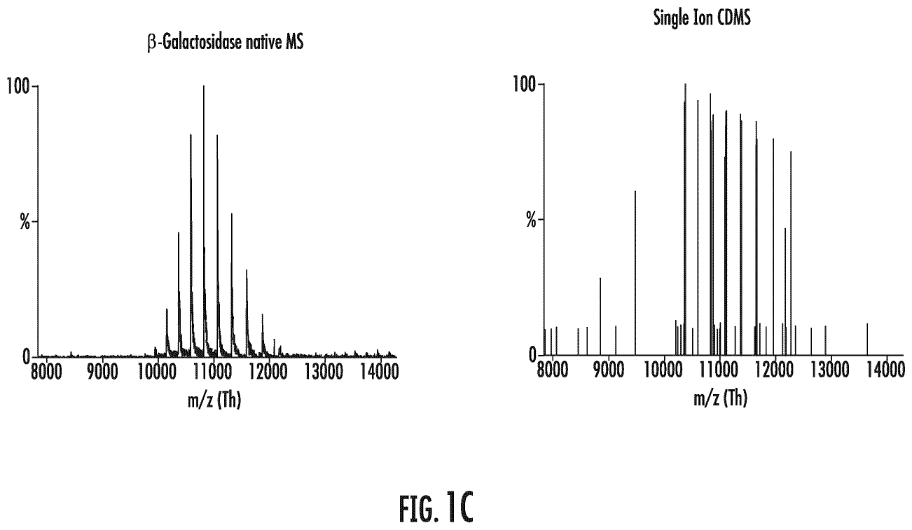

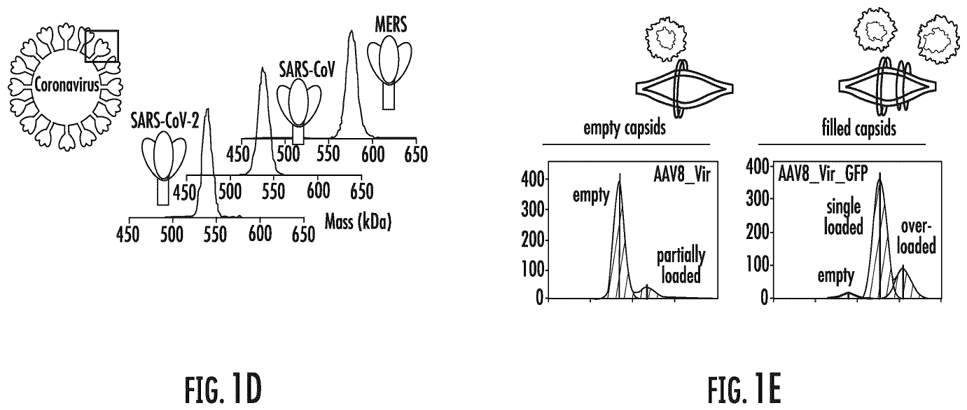

The purity of AAV products is critical for improved transduction and efficacy of the product. Therefore, it is essential to have an analytical method that can provide quantitative information about the empty/partially filled/full distribution (also called “capsid content”) of AAV capsids. One method that has been used for AAV vector characterization is charge detection mass spectrometry (CDMS). CDMS is a single ion technique that enables the direct mass measurement of ions by simultaneously measuring the frequency (or mass-to-charge ratio, m/z) and charge (z) of ions. These individual single ion measurements are then mass binned and plotted as a mass domain spectrum, as illustrated in FIG. 1B-FIG. 1C. Mass can be directly calculated by multiplying z and m/z. FIG. 1A depicts an example schematic diagram of an Orbitrap analyzer for charge-detection mass spectrometry (CDMS) analysis.

CDMS has proven particularly powerful for evaluating the mass of large and/or highly heterogeneous samples, such as AAV vectors, e.g., full, partial filled, or empty AAV capsids, where individual charge states cannot be resolved, thus rendering conventional MS based techniques incapable of mass interpretation, as illustrated in FIG. 1D and FIG. 1E. Specifically with regard to Orbitrap-based CDMS, image current detection improves the efficiency of single ion collection, multiplexing single ion detection to one ion at a defined frequency per acquisition. Long time-domain signals are subject to ion decay, resulting in decreased signal amplitude, which can lead to underestimation of z and mass. Selective Temporal Overview of Resonant Ions (STORI) analysis can be used to determine z using the rate of signal accumulation instead of its total amount.

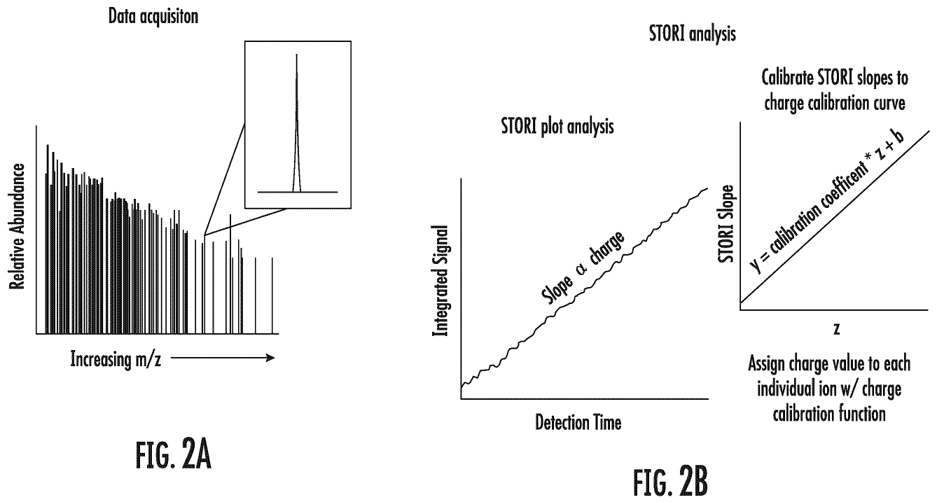

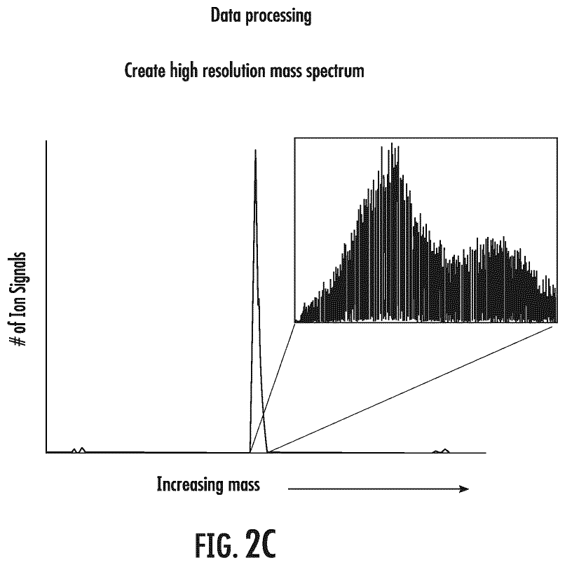

In practice, Orbitrap-based CDMS may be achieved through Direct Mass Technology (DMT) (Kafader et al., 2020, Nat. Methods, 17(4):391-394) using STORI analysis. First, single ion data is collected, as shown on FIG. 2A. Hundreds of single ions per spectrum in m/z space are collected. The frequencies and amplitudes of individual ions are simultaneously measured in DMT. Next, each ion is analyzed using a STORI plot, as shown in FIG. 2B. The induced current signal is integrated and plotted over time. The STORI slope of an individual ion is proportional to z. A charge calibration function is used to calibrate STORI slopes. Afterward, the data is processed as shown in FIG. 2C. The mass domain spectrum is calculated, with the mass of each ion determined by multiplying m/z and z. The masses are binned into a histogram (CDMS spectrum).

One important consideration for CDMS analysis is that to generate meaningful and statistically significant mass domain spectra, hundreds-to-thousands of single ion measurements are typically required. For large and highly complex samples, this may require acquisition periods of 30 minutes to several hours, which in turn demands stable and robust sample infusion over the course of the experiment. Currently, many researchers rely on static nano infusion, or some variation thereof, for sample introduction for CDMS acquisition. Using static infusion, AAV signal is detected, but unstable. While static infusion has the benefit of no sample dilution and high sensitivity, this method 1) involves an expert/experienced operator, 2) lacks robustness and can be subject to frequent tip clogging, and 3) involves offline buffer exchange into MS compatible buffers prior to analysis. Therefore, improved methods are needed for reliable and efficient analysis of viral vector heterogeneity.

In order to address these long-felt needs, this disclosure sets forth a SEC-FI platform that streamlines and automates the sample introduction method. The developed platform uses a dual high-flow/low-flow configuration that enables fast online size exclusion-based desalting and buffer exchange under high flow conditions, as well as stable and long-lasting sample infusion with limited dilution under low flow conditions. Additionally, this dual flow configuration was coupled with a MnESI source equipped with a multinozzle M3 emitter for stable, robust, and sensitive nano-infusion. These systems and methods are particularly powerful for Orbitrap-based CDMS analysis of AAV, as shown in the Examples below.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The terms “about” in the context of numerical values and ranges refers to values or ranges that approximate or are close to the recited values or ranges such that the invention can perform as intended, such as having a desired rate, amount, density, degree, increase, decrease, percentage, ratio, value, purity, pH, concentration, presence of a form or variant, temperature or amount of time, as is apparent from the teachings contained herein. For example, “about” can signify values either above or below the stated value in a range of +/−10% or more or less depending on the ability to perform. Thus, this term encompasses values beyond those simply resulting from systematic error.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. As used herein, the term polypeptide includes proteins, variants thereof, fragments thereof, and peptides, whether synthetic, naturally occurring, or derived from a larger polypeptide, for example through digestion or truncation. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like.

Proteins of interest or polypeptides of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference).

In some exemplary aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of. IgG, IgM, IgAQ1, IgA2, IgD, or IgE. In certain aspects the antibody molecule is a full-length antibody (e.g., an IgG1) or alternatively the antibody can be a fragment (e.g., an Fc fragment or a Fab fragment).

The term “antibody” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a Fc/2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen.

An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KX-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the systems and methods disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, a “sample” can be obtained from any step of a bioprocess, such as cell culture fluid (CCF), harvested cell culture fluid (HCCF), any step in the downstream processing, final concentrated pool (FCP), drug substance (DS), or a drug product (DP) comprising the final formulated product. In some specific aspects, the sample can be selected from any step of the downstream process of clarification, chromatographic production, or filtration. In some specific exemplary aspects, the drug product can be selected from manufactured drug product in the clinic, shipping, storage, or handling.

In some exemplary aspects, the sample is a biological sample. As used herein, the term “biological sample” refers to a sample taken from a living organism, for example a human or non-human mammal. A biological sample may comprise or consist of, for example, whole blood, plasma, serum, saliva, tears, semen, cheek tissue, organ tissue, urine, feces, skin, or hair. A sample may be taken from a patient, for example, a clinical sample. In some exemplary aspects, a sample may be taken from a non-human animal, for example, a preclinical sample. In some exemplary aspects, a sample may be taken from a non-human animal subjected to gene therapy in order to produce at least one protein of interest or polypeptide of interest that may be included in the sample. In some aspects, a sample is a further processed form of any of the aforementioned examples of samples.

The following identifies and describes proteins made in cell culture that can be produced and/or characterized according to the present inventions. Cells comprising the requisite DNA encoding these proteins can be cultured for production according to the present inventions.

In some exemplary aspects, the protein of interest or polypeptide of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

For example, for antibody production, some embodiments are amenable for research and production use for diagnostics and therapeutics based on all major antibody classes, namely IgG, IgA, IgM, IgD, and IgE. IgG is a preferred class, and includes subclasses IgG1 (including IgG1k and IgG1x), IgG2, IgG3, and IgG4. In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. Derivatives, components, domains, chains, and fragments of the above are also included.

In some aspects, the antibody is selected from the group consisting of an anti-Programmed Cell Death 1 antibody (e.g. an anti-PD1 antibody as described in U.S. Pat. App. Pub. No. US2015/0203579A1), an anti-Programmed Cell Death Ligand-1 antibody (e.g. an anti-PD-L1 antibody as described in in U.S. Pat. App. Pub. No. US2015/0203580A1), an anti-DII4 antibody, an anti-Angiopoietin-2 antibody (e.g. an anti-ANG2 antibody as described in U.S. Pat. No. 9,402,898), an anti-Angiopoietin-Like 3 antibody (e.g. an anti-AngPtl3 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. App. Pub. No US2015/0313194A1), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. App. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. App. Pub. No. US2014/0044730A1), an anti-Growth And Differentiation Factor-8 antibody (e.g. an anti-GDF8 antibody, also known as anti-myostatin antibody, as described in U.S. Pat. No. 8,871,209 or 9,260,515), an anti-Glucagon Receptor (e.g. anti-GCGR antibody as described in U.S. Pat. App. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. App. Pub. No. US2014/0271681A1 or U.S. Pat. No. 8,735,095 or 8,945,559), an anti-interleukin 6 receptor antibody (e.g. an anti-IL6R antibody as described in U.S. Pat. No. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. App. Pub. Nos. US2014/0271658A1 or US2014/0271642A1), an anti-Cluster of differentiation 3 antibody (e.g. an anti-CD3 antibody, as described in U.S. Pat. App. Pub. Nos. US2014/0088295A1 and US20150266966A1, and in U.S. Application No. 62/222,605), an anti-Cluster of differentiation 20 antibody (e.g. an anti-CD20 antibody as described in U.S. Pat. App. Pub. Nos. US2014/0088295A1 and US20150266966A1, and in U.S. Pat. No. 7,879,984), an anti-CD19 antibody, an anti-CD28 antibody, an anti-Cluster of Differentiation-48 antibody (e.g. anti-CD48 antibody as described in U.S. Pat. No. 9,228,014), an anti-Fel d1 antibody (e.g. as described in U.S. Pat. No. 9,079,948), an anti-influenza virus antibody, an anti-Respiratory syncytial virus antibody (e.g. anti-RSV antibody as described in U.S. Pat. App. Pub. No. US2014/0271653A1), an anti-Middle East Respiratory Syndrome virus antibody (e.g. an anti-MERS-CoV antibody as described in U.S. Pat. App. Pub. No. US2015/0337029A1), an anti-Ebola virus antibody (e.g. as described in U.S. Pat. App. Pub. No. US2016/0215040), an anti-Zika virus antibody, an anti-Severe Acute Respiratory Syndrome (SARS) antibody (e.g., an anti-SARS-CoV antibody), an anti-COVID-19 antibody (e.g., an anti-SARS-CoV-2 antibody), an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. App. Pub. No. US2016/0017029 and U.S. Pat. Nos. 8,309,088 and 9,353,176) and an anti-Activin A antibody. In some aspects, the bispecific antibody is selected from the group consisting of an anti-CD3 xanti-CD20 bispecific antibody (as described in U.S. Pat. App. Pub. Nos. US2014/0088295A1 and US20150266966A1), an anti-CD3×anti-Mucin 16 bispecific antibody (e.g., an anti-CD3×anti-Mucl6 bispecific antibody), an anti-CD3×BCMA bispecific antibody, and an anti-CD3×anti-Prostate-specific membrane antigen bispecific antibody (e.g., an anti-CD3×anti-PSMA bispecific antibody). See also U.S. Patent Publication No. US 2019/0285580 A1. Also included are a Met×Met antibody, an agonist antibody to NPR1, an LEPR agonist antibody, a MUC16×CD28 antibody, a GITR antibody, an IL-2Rg antibody, an EGFR×CD28 antibody, a Factor XI antibody, antibodies against SARS-CoV-2 variants, a Fel d 1 multi-antibody therapy, and a Bet v 1 multi-antibody therapy. Derivatives, components, domains, chains and fragments of the above also are included. In one aspect, the protein of interest or polypeptide of interest comprises a combination of any of the foregoing.

Cells that produce exemplary antibodies can be cultured according to the inventions. In some aspects, the protein of interest or polypeptide of interest is selected from the group consisting of Alirocumab, Atoltivimab, Maftivimab, Odesivimab, Odesivimab-ebgn, Casirivimab, Imdevimab, Cemplimab and Cemplimab-rwlc (human IgG4 monoclonal antibody that binds to PD-1), Sarilumab, Fasinumab, Nesvacumab, Dupilumab (human monoclonal antibody of the IgG4 subclass that binds to the IL-4R alpha (a) subunit and thereby inhibits Interleukin 4 (IL-4) and Interleukin 13 (IL-13) signaling), Trevogrumab, Evinacumab, Evinacumab-dgnb, Fianlimab, Garetosmab, Itepekimab, Odrononextamab, Pozelimab, Rinucumab, and modifications, truncations, and variations thereof.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

In addition to next generation products, the invention also applies to production of biosimilars. Biosimilars are described in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to a reference product already approved in Japan. In India, biosimilars currently are referred to as “similar biologics,” and refer to a similar biologic product which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, a biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be undertaken according to the inventions.

In some aspects, the protein of interest or polypeptide of interest is a recombinant protein that contains an Fc moiety and another domain, (e.g., an Fc-fusion protein). In some aspects, an Fc-fusion protein is a receptor Fc-fusion protein, which contains one or more extracellular domain(s) of a receptor coupled to an Fc moiety. In some aspects, the Fc moiety comprises a hinge region followed by a CH2 and CH3 domain of an IgG. In some aspects, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the II-1R1 extracellular region fused to Fc of hIgG1; see U.S. Pat. No. 6,927,004, which is herein incorporated by reference in its entirety), or a VEGF trap (e.g., aflibercept or ziv-aflibercept, which contains the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). In other aspects, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

In one aspect, the protein of interest comprises a combination of any of the foregoing.

As used herein, the term “vector” refers to a recombinant plasmid or virus (“viral vector”) that comprises a nucleic acid to be delivered into a host cell, either in vitro or in vivo. Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, for example, interferon-mediated responses; (iii) wild type AAVs have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

A “recombinant AAV vector (rAAV vector)” refers to a polynucleotide vector including one or more heterologous sequences (i.e., nucleic acid sequence not of AAV origin) that may be flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins).

A “capsid” is the protein shell of a virus, which encloses the genetic material. Three viral capsid proteins, VP1, VP2 and VP3, form the viral icosahedral capsid of 60 subunits in a ratio of 1:1:10. A full capsid contains genetic material and is required to provide therapeutic benefit. An empty capsid lacks the genome and therefore lacks the ability to provide therapeutic benefit to the patient.

A “viral particle” refers to a particle composed of at least one viral capsid and an encapsulated viral genome. While AAV is described in this disclosure as a model virus or viral particle, it is contemplated that the disclosed methods can be applied to profile a variety of viruses, e.g., the viral families, subfamilies, and genera. In some aspects, the viral capsid, virus, or viral particle belongs to a viral family selected from the group consisting of Adenoviridae, Parvoviridae, Retroviridae, Baculoviridae, and Herpesviridae. In some aspects, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roseolovirus, Lymphocryptovirus, Macavirus, Percavirus, and Rhadinovirus.

In some aspects, a sample can be prepared prior to or following enrichment steps, separation steps, and/or analysis steps. Preparation steps can include alkylation, reduction, denaturation, digestion, derivatization, and/or deglycosylation.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some aspects, non-reduced preparation may be used, for example, in order to preserve an endogenous disulfide bond between Fab arms of an antibody or antibody-derived protein. In other aspects, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein or polypeptide. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein or polypeptide. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), IdeZ, IgdE, glyserias, thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary aspects, IdeS or a variant thereof is used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Alternatively, or additionally, digestion that separates out a Fab₂ fragment and keeps an Fc fragment for analysis may be preferred, because the Fc fragment contains an N-glycosylation site of interest.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FabRICATOR® or FabRICATOR Z®.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC). In some aspects, a sample can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

In some exemplary aspects, the methods and systems of the present invention include the use of size exclusion chromatography. Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

In some exemplary aspects, the mobile phase used to obtain an eluate from size exclusion chromatography can comprise a volatile salt. In some specific aspects, the mobile phase can comprise ammonium acetate, ammonium bicarbonate, or ammonium formate, or combinations thereof.

Online coupling of SEC with direct MS detection under near native conditions (native SEC-MS) has gained interest in recent years to study mAb HMW species (Rouby et al., supra; Ehkirch A, Hernandez-Alba 0, Colas 0, Beck A, Guillarme D, Cianferani S. Hyphenation of size exclusion chromatography to native ion mobility mass spectrometry for the analytical characterization of therapeutic antibodies and related products. J Chromatogr B Analyt Technol Biomed Life Sci 2018:1086 (176-183); Haberger M, Leiss M, Heidenreich A K, Pester 0, Hafenmair G, Hook M, Bonnington L, Wegele H, Haindl M, Reusch D et al. Rapid characterization of biotherapeutic proteins by size-exclusion chromatography coupled to native mass spectrometry. MAbs 2016:8(2): 331-339. Using MS-compatible mobile phases that can preserve protein conformation and non-covalent interactions, native SEC-MS (nSEC-MS) can provide rapid and improved identification of size variants or similarly sized polypeptides in a sample based on accurate mass measurement.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary aspects, the mass spectrometer may use a microflow nanospray ion source, for example Newomics® MnESI (microflow nanospray electrospray ionization) ion source, with M3 emitter. The ion source has multiple nozzles working together to split a single microflow stream evenly into multiple nanoflows. The MnESI source can be linked to a high-flow LC system using a T-splitter. This configuration allows a microflow to enter the MnESI, while the remaining analytical flow is directed towards an additional detector or system.

In some aspects, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion is selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some aspects, LC-MS can be performed under native conditions. As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution. Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. In some aspects, native MS allows for better spatial resolution compared to non-native MS. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE pp. 1176-1192 (2015).

Examples have been set forth below for the purpose of illustration and to describe certain specific embodiments of the invention. However, the scope of this invention is not to be in any way limited by the examples set forth herein.

### EXAMPLES

All AAV samples were produced at Regeneron Pharmaceuticals, Inc. (Tarrytown, NY). Deionized water used in sample and buffer preparations was provided by a Milli-Q integral water purification system equipped with a MilliPak Express 20 filter (MilliporeSigma, Burlington, MA). Ammonium acetate (LC-MS grade) was purchased from Sigma-Aldrich (St. Louis, MO). Native SEC chromatography was performed on an UltiMate 3000 UHPLC System (Thermo Fisher Scientific, Bremen, Germany) equipped with an AcquityBEH200 SEC column (1.0×50 mm, 1.7 μm, 200 Å; Waters, Milford, MA) with the column compartment set to 30 °C. A Thermo Q Exactive UHAMR (Thermo Fisher Scientific, Bremen, Germany) equipped with a microflow-nanospray electrospray ionization (MnESI) source and a microfabricated monolithic multinozzle (M3) emitter (Newomics, Berkley, CA) was used for native MS and CDMS analysis.

AAVs were first online exchanged by the SEC column into 150 mM ammonium acetate mobile phase at a high flow rate (20 μL/min). To enable online native MS analysis, the combined analytical flow (20 μL/min) was split into a microflow (<10 μL/min) for electrospray ionization (ESI)-MS detection. To enable online CDMS analysis, a sample loop was added to trap the desalted analytes followed by nano-ESI spray achieved at a low flow rate (1 μL/min) using a secondary pump. Online SEC-CDMS analysis of the protein standards was performed in a similar configuration using an Acquity BEH200 SEC guard column (4.6×150 mm, 1.7 μm, 200 Å) and 200 μL sample loop, or an optimized column (1.0×50 mm) and 60 μL sample loop. CDMS spectra were acquired using Direct Mass Technology (DMT) and processed using Proteinaceous STORIBoard with frequency correction function developed by Thermo Fisher Scientific.

### Example 1. Size Exclusion Chromatography-Flow Injection for Charge-Detection Mass Spectrometry Injection

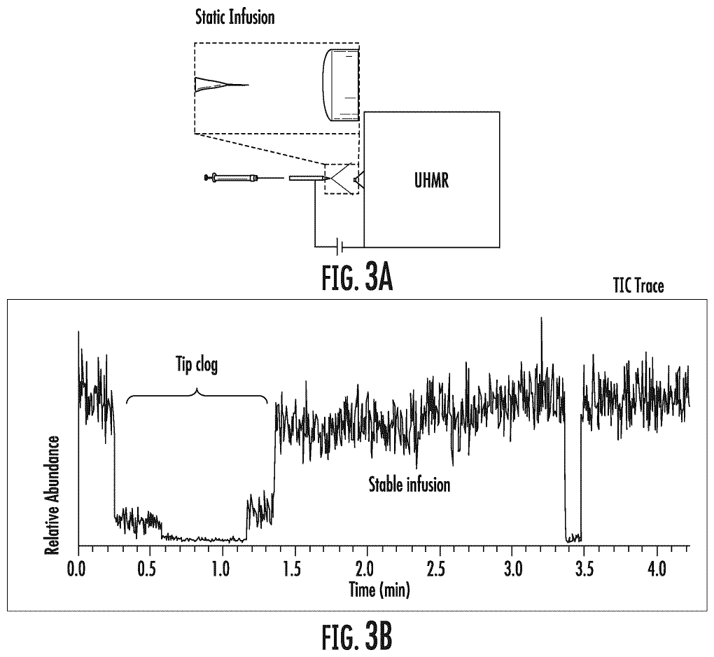

Charge-detection mass spectrometry (CDMS) typically involves hundreds to thousands of single ion measurements to generate statistically significant spectra, which in turn requires stable sample infusion for 30 minutes to several hours. Using static infusion of an AAV sample, AAV signal was detected, but unstable. Static infusion involves an expert operator, lacks robustness, can be subject to frequent tip clogging, and involves offline buffer exchange into MS-compatible buffers prior to analysis, which likely leads to AAV samples precipitating and aggregating in solution. An example of a static infusion system is shown in FIG. 3A, and a corresponding total ion chromatogram (TIC) trace is shown in FIG. 3B.

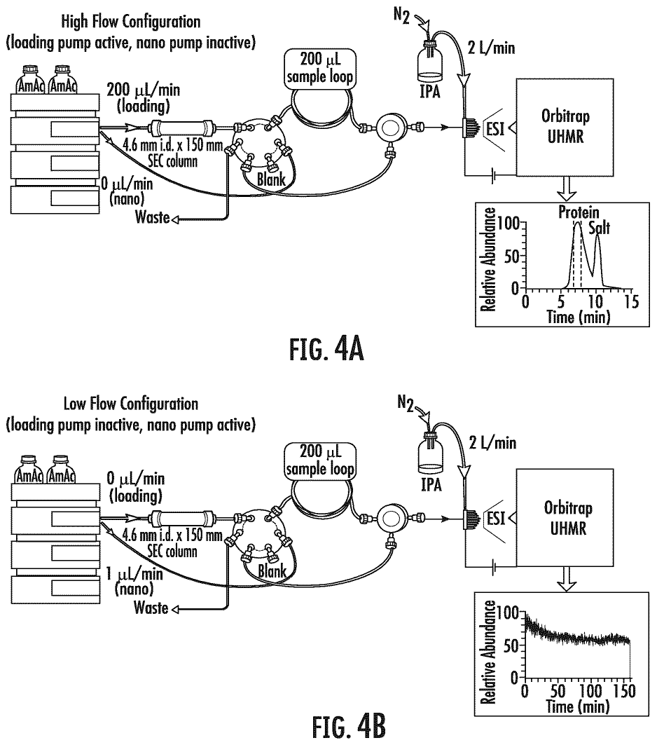

Two desirable features for improving a sample introduction platform for CDMS analysis of AAVs include a platform with online size-exclusion-based desalting and buffer exchange, and stable and long-lasting sample infusion with limited dilution. To this end, size exclusion chromatography-flow injection (SEC-FI) methods and systems were developed, as illustrated in FIG. 4A and FIG. 4B. The system includes a microflow nanospray ion source equipped with a multinozzle M3 emitter for stable, robust, and sensitive nano-infusion. Both a high-flow configuration and a low-flow configuration are possible with the developed system.

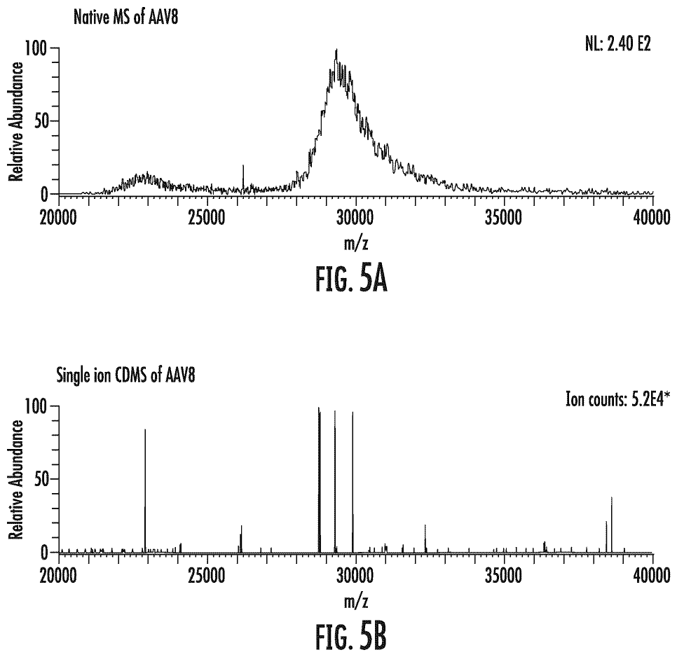

The high-flow configuration allows for efficient online desalting, with no buffer exchange required. Size exclusion-based online desalting and native MS measurement can be used to quickly assess the full/empty ratio of AAV vectors. The loading pump is coupled to the SEC column. The desalted sample can be trapped in an extended sample loop. A survey run is used to establish the timing of the valve switch to trap desalted sample in the extended loop. The effectiveness of online desalting of an AAV sample using the high-flow SEC-FI-MS system is shown in a native mass spectrum (MS) in FIG. 5A.

The low-flow configuration allows for stable and long-lasting sample infusion in the range of about 30 minutes to several hours with minimal sample dilution, thus allowing Orbitrap-based CDMS analysis. The nano pump is directly coupled to the sample loop and facilitates stable spray at flow rates as low as 1 μL/minute. As many as 200 minutes of infusion time are possible. The stability of injection of an AAV sample using the low-flow SEC-FI-MS system is shown in a CDMS in FIG. 5B.

### Example 2. Optimizing the SEC-FI-MS Systems and Methods

Using the SEC-FI-MS system and method described above, it was determined that both native MS signal intensity and CDMS ion counts were relatively low, and only few single ions were collected at the maximum injection time. This suggested that there was significant sample dilution. Therefore, the systems and methods were further optimized.

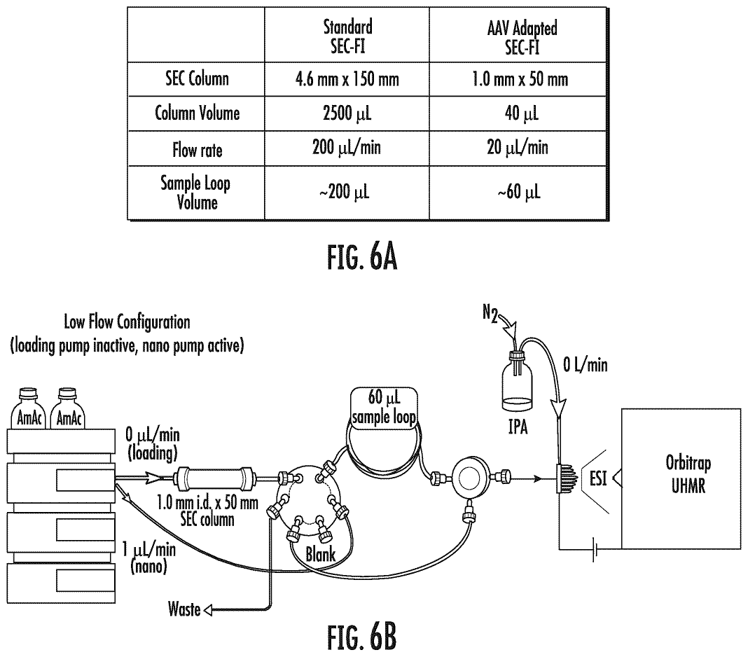

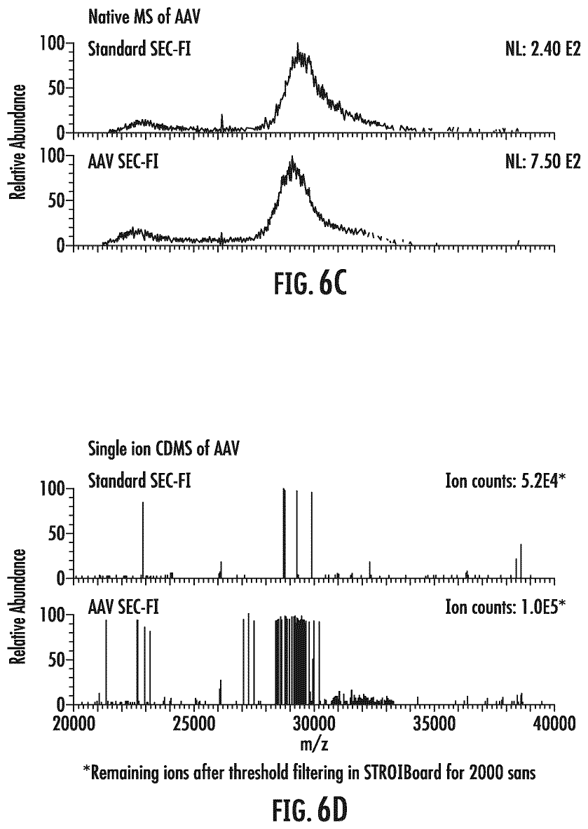

To reduce sample dilution, a new SEC column with a smaller volume and a shorter sample loop were used (“AAV Adapted SEC-FI”), as shown in FIG. 6A and FIG. 6B. Using the optimized platform, a similar desalting result was achieved while the signal intensity of AAVs was increased in both native MS and single ion CDMS, as shown in FIG. 6C and FIG. 6D. The effective ion counts were doubled compared to the previous configuration.

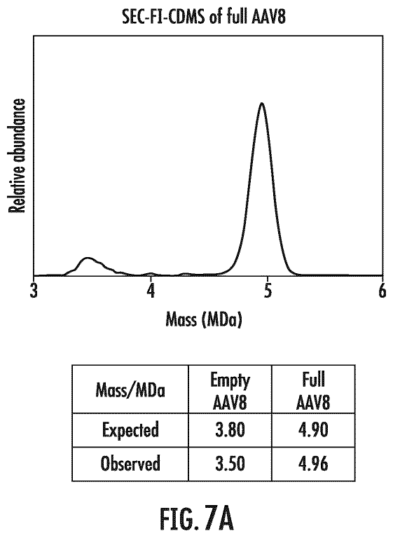

Additionally, while it was possible to detect sufficient CDMS signal for AAVs using the SEC-FI-MS platform described in Example 1, the quality of these scans was not certain. The AAV mass measured using SEC-FI was sometimes observed to be significantly lower than the expected mass, as shown in FIG. 7A. To determine the reason for the mass difference, individual ion spectra were checked, and it was found that the peak shapes were badly distorted compared to those from static infusion. This suggests that there is frequency shifting during Orbitrap measurement, which is likely due to the higher amount of solvation in SEC-FI.

To generate high-quality CDMS spectra, source conditions were optimized, and the sheath gas was identified as a key factor. After removing the sheath gas, the peak shape of a single ion was greatly improved to nearly Gaussian distribution, though the peak was still asymmetric, indicating that some frequency shifting remained, as shown in FIG. 7B.

The frequency shifting issue was further investigated. It has been shown that the STORI method can underestimate signals for ions with unstable frequencies. A frequency correction function has been described to correct the frequency difference between real signal and STORI slope. Briefly, this is achieved by determining and fitting the frequency difference in the time derivatives of the STORI plot. The variable frequency changes over time are calculated in many linear segments and then are continuously fed back into the STORI calculation to correct the STORI frequency.

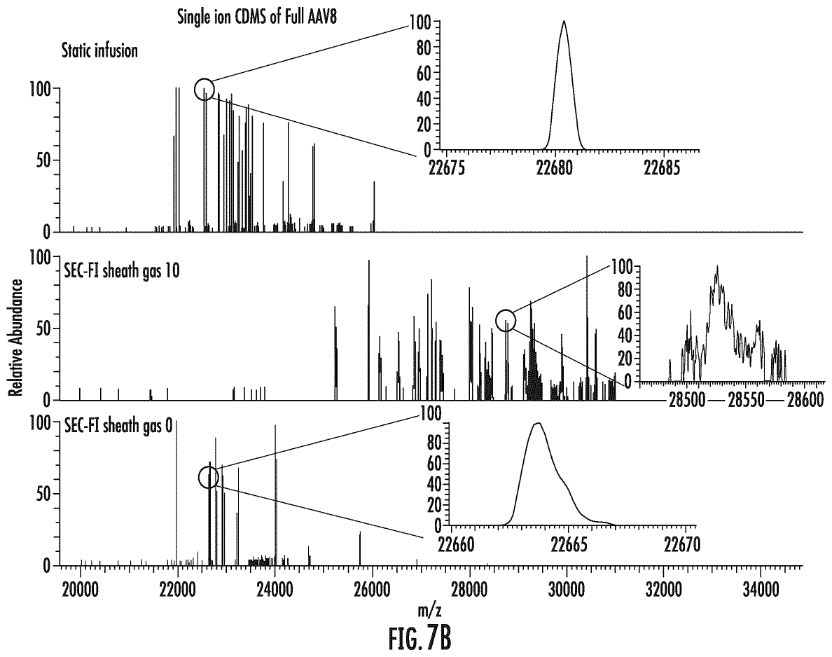

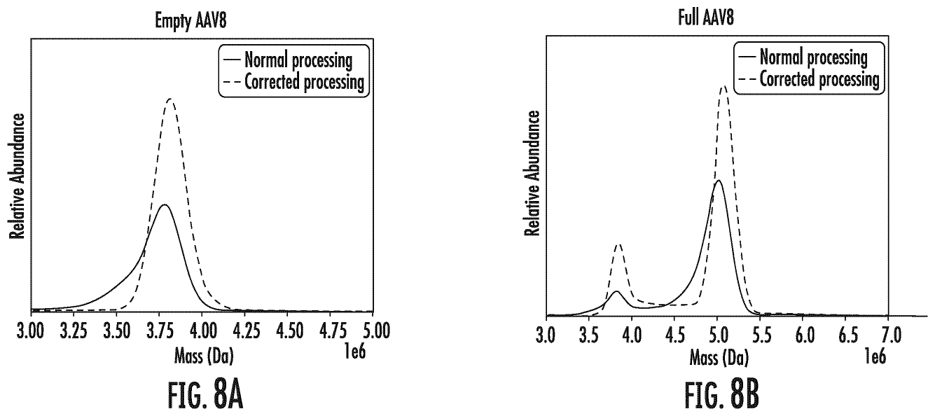

The frequency correction function was applied to the SEC-FI-MS AAV analysis, allowing for accurate AAV mass calculations. Additionally, more ions were retained and peaks were better resolved after the corrective signal processing, as shown in FIG. 8A and FIG. 8B.

### Example 3. Validation of SEC-FI-CDMS for AAV Analysis

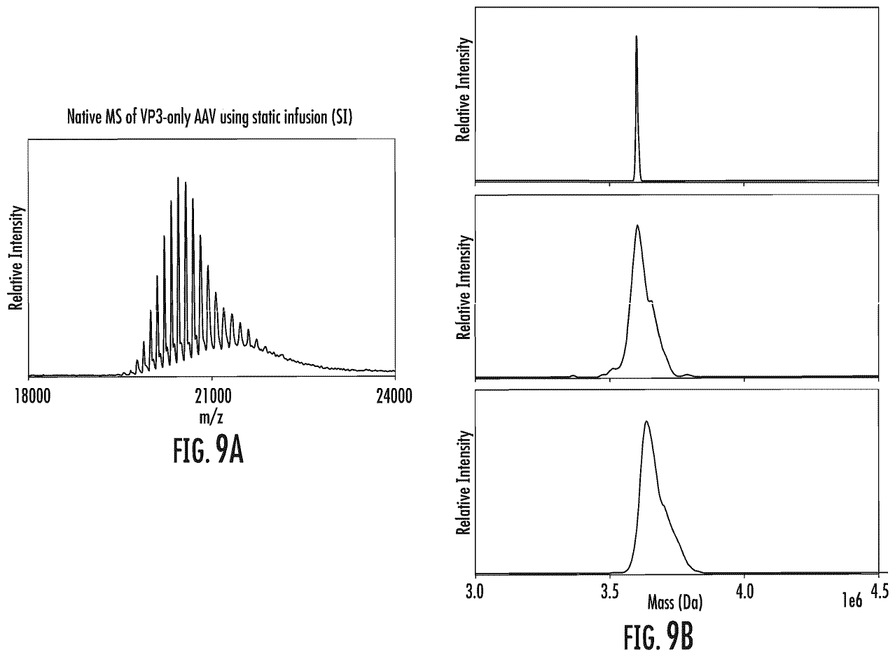

As a proof of concept, AAVs containing only VP3 protein were evaluated using the SEC-FI-CDMS platform. AAVs containing only VP3 are relatively homogeneous, so charge states can be readily resolved using native MS spectra. The mass determined by SEC-FI-CDMS was higher than those from native MS and CDMS using static infusion, suggesting that there are higher amounts of solvation, as shown in FIG. 9A and FIG. 9B.

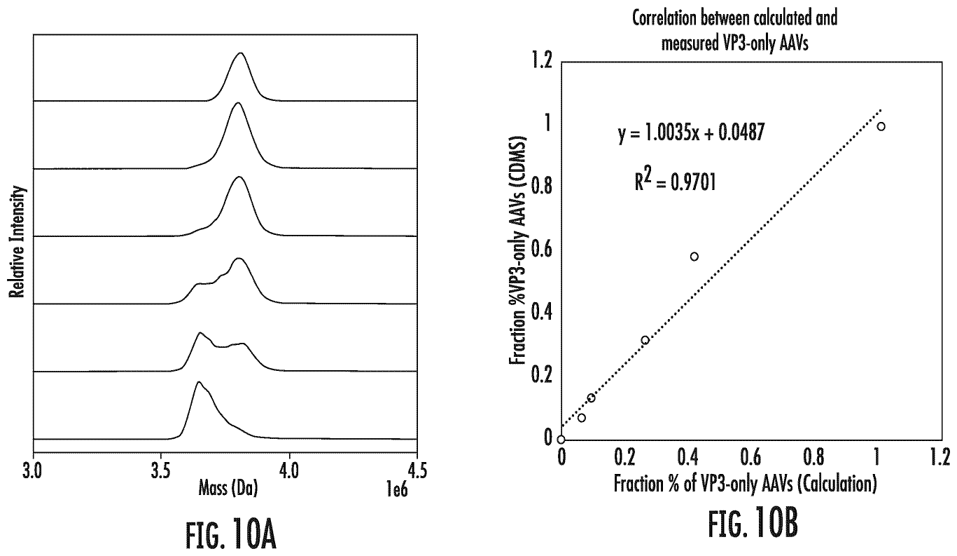

The resolution of MS detection using SEC-F1 was evaluated for both native MS and CDMS. The VP3-only AAV sample was spiked into an empty AAV8 control lot at a series of mixing ratios and analyzed using the SEC-FI-CDMS platform. The presence of VP3-only AAVs could be identified at the lowest level tested, 7.3%, as shown in FIG. 10A and FIG. 10B.

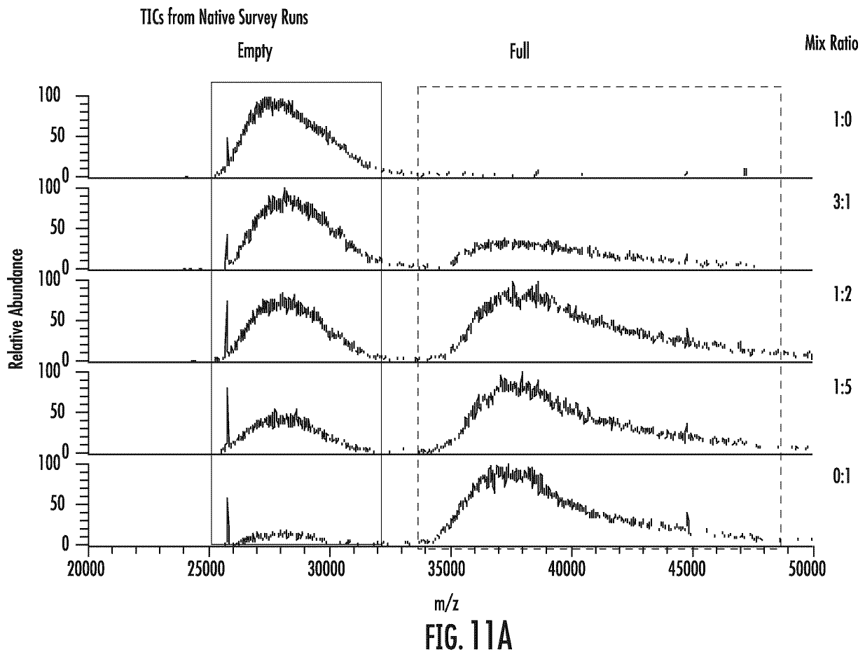

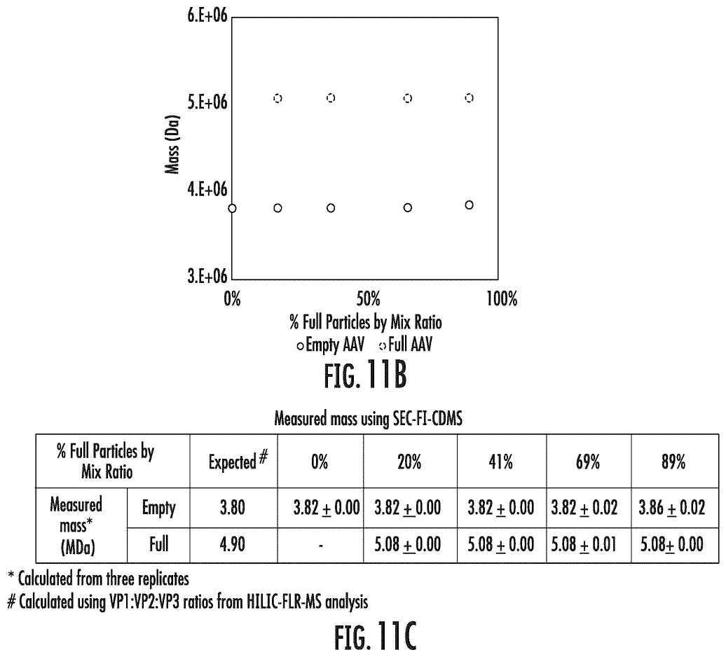

In order to validate accuracy and reproducibility of the developed SEC-FI-MS system and method, the method was performed using lot material of a full AAV8 vector, spiked into an AAV8 control lot with only empty capsid, as shown in FIG. 11A. In this manner, a variety of mixtures of empty and full capsids were produced, with the full capsid percentage ranging from 0% to 89%. A native MS survey scan was performed for each sample using high flow SEC-FI. Samples were quantified by directly integrating native MS spectra at m/z 26-31 k for empty capsids and 33-48 k for full capsids.

The CDMS measured masses of empty and full capsids aligned with predicted values across all mixtures, as shown in FIG. 11B and FIG. 11C. The CDMS measurement of empty and full AAV8 could be consistently reproduced within 0.2% mass difference, as shown in FIG. 11D. The measured empty/full ratios (E:F) were in good agreement with those calculated using analytical ultracentrifugation (AUC) (y=1.1515x−0.1112; R²=0.9852), as shown in Table 1. Overall, the high reproducibility and precision of the optimized SEC-FI-CDMS platform was confirmed.

TABLE 1

| Mixing ratio E:F | Calculated full (AUC) | Measured full (MS) |
|---|---|---|
| 1:0 | 0 | 0.07 |
| 3:1 | 0.20 | 0.33 |
| 1:2 | 0.56 | 0.58 |
| 1:5 | 0.72 | 0.68 |
| 0:1 | 0.89 | 0.88 |

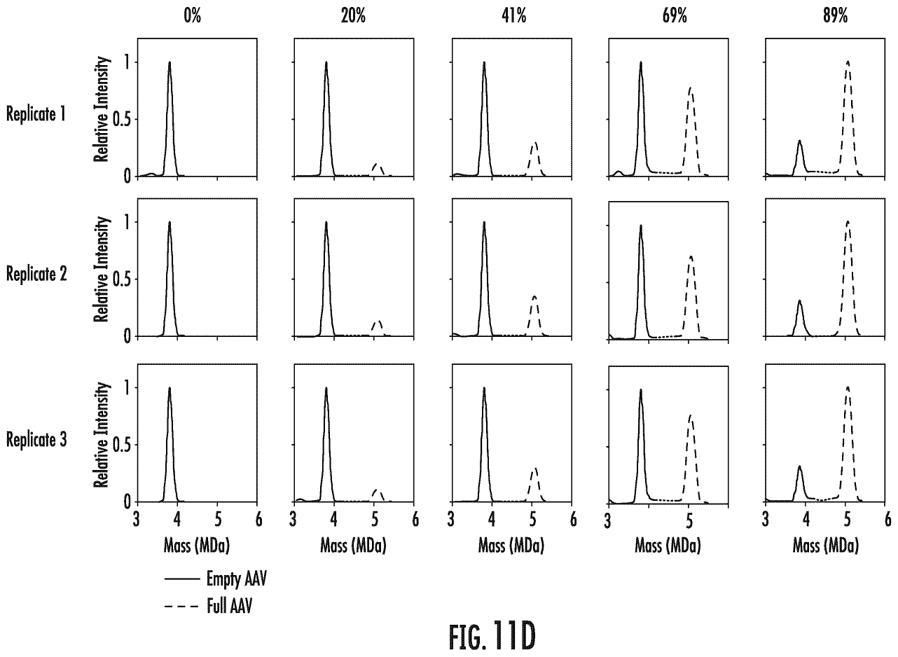

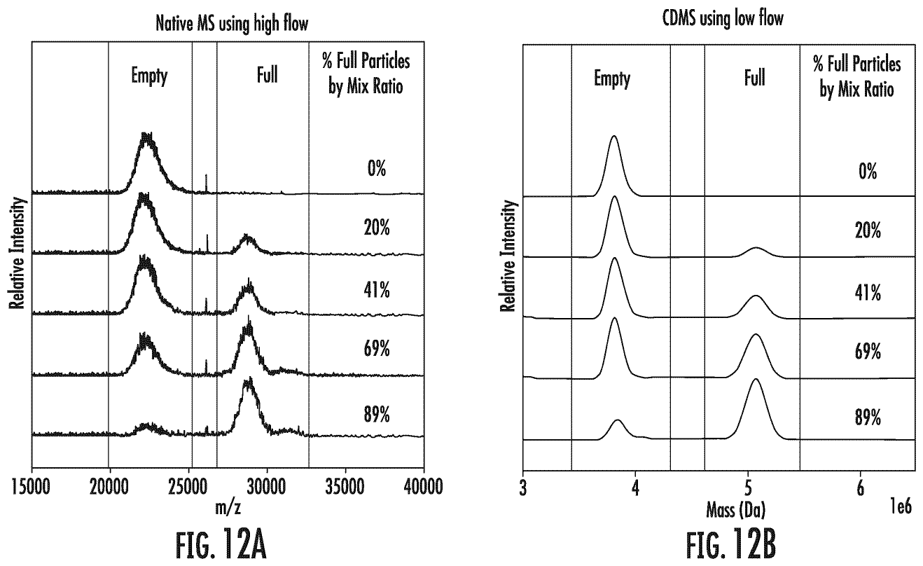

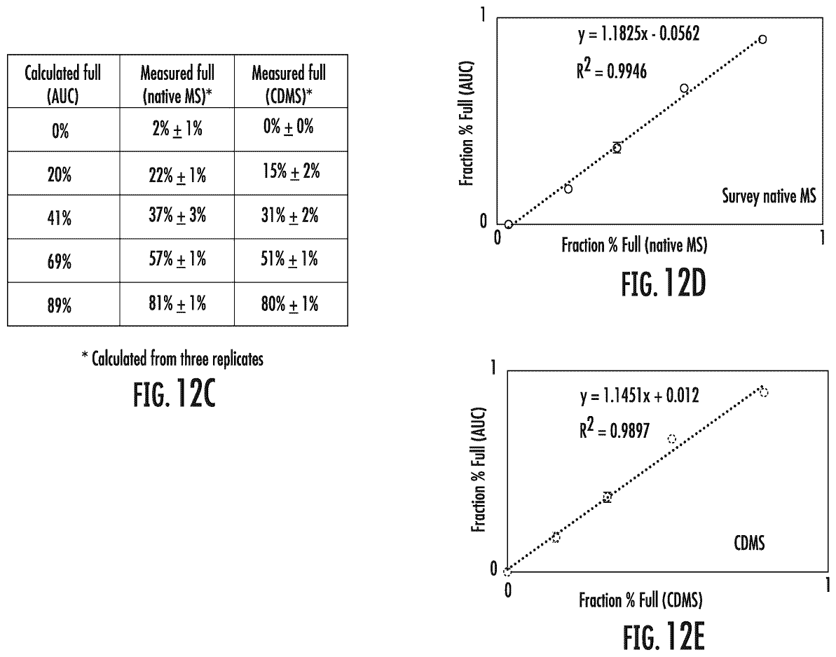

Next, the quantification performance of the platform was assessed for determining E:F ratios using the same mixed samples described above. The relative abundance of capsids determined from m/z range in the native MS spectra using the high flow configuration aligned well with that calculated from the CDMS spectra using the low flow configuration, as shown in FIG. 12A and FIG. 12B. Additionally, the measured E:F ratios from the two FI configurations showed good linear relationships with those calculated from analytical ultracentrifugation (AUC), which is often considered the gold standard for AAV quantification, as shown in FIG. 12C-FIG. 12E. This test demonstrated that the developed platform can provide effective quantification for AAVs in addition to accurate mass measurement.

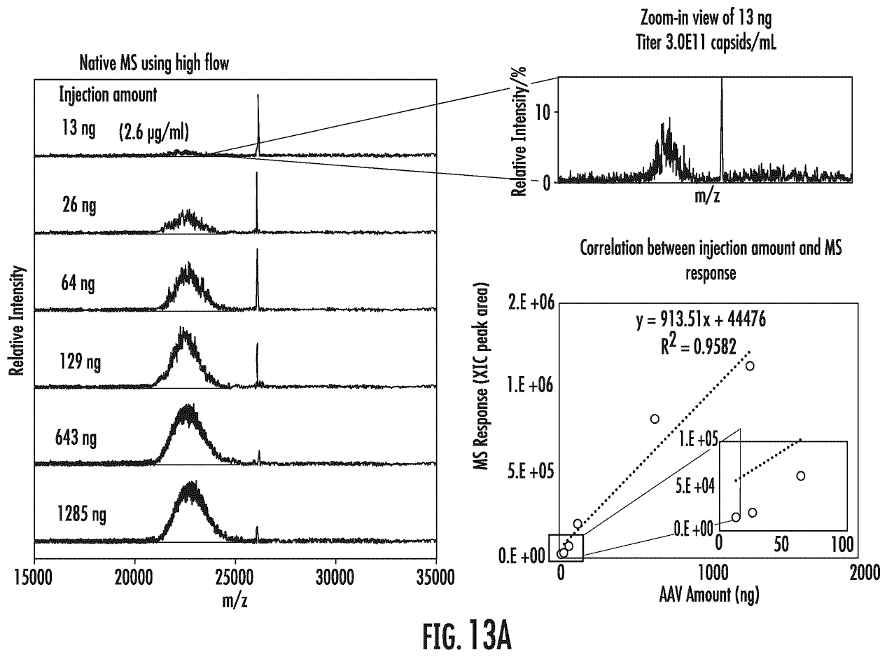

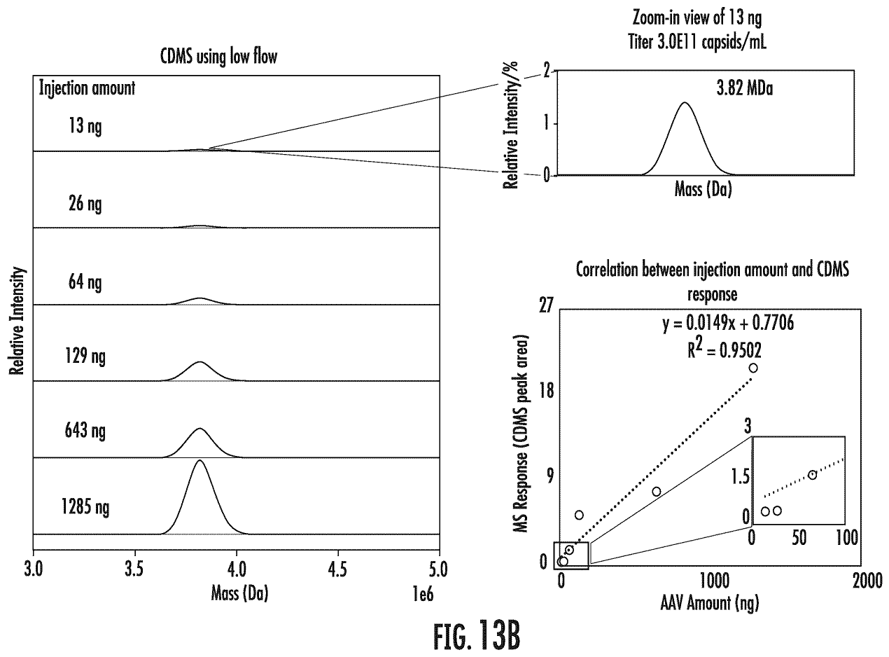

The platform was additionally evaluated for the ability to detect and analyze AAVs even at low concentrations. This was achieved by varying the injection amount of empty AAV8 capsids. Both native MS and CDMS responses showed a strong linear relationship to the injection amount, as shown in FIG. 13A and FIG. 13B. Masses for empty AAV8 capsids could be detected and accurately assigned even at a low titer of 3.OE11 capsids/mL. Therefore, the platform demonstrated high sensitivity suitable for application to regular AAV programs.

### Example 4. Application of SEC-FI-CDMS for AAV Analysis

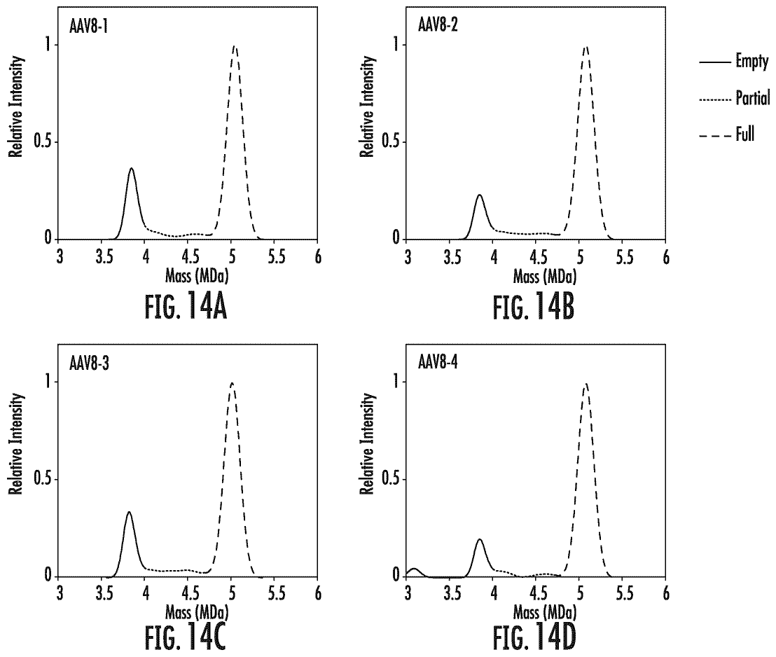

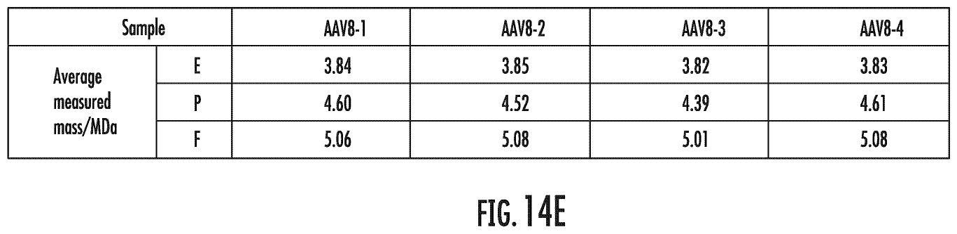

Orbitrap-based SEC-FI-CDMS analysis with frequency correction was applied for the analysis of AAV vectors. Four lots of the AAV8 vector, labeled AAV8-1 to AAV8-4, were subjected to low-flow SEC-FI-CDMS, as shown in FIG. 14A-FIG. 14D respectively. Empty, partially-filled, and full vectors were separated, and the average mass of each was measured, as shown in FIG. 14E.

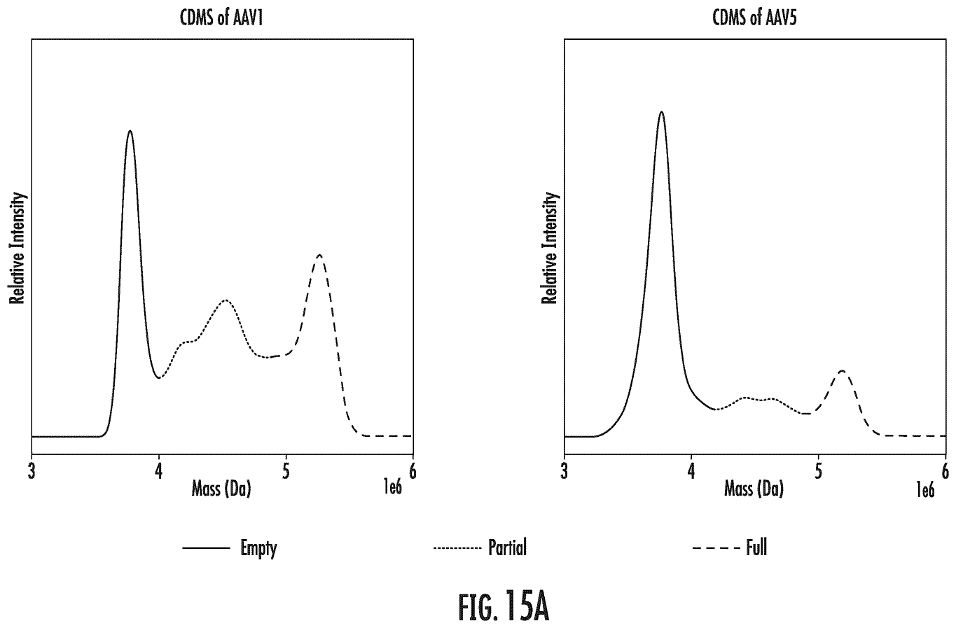

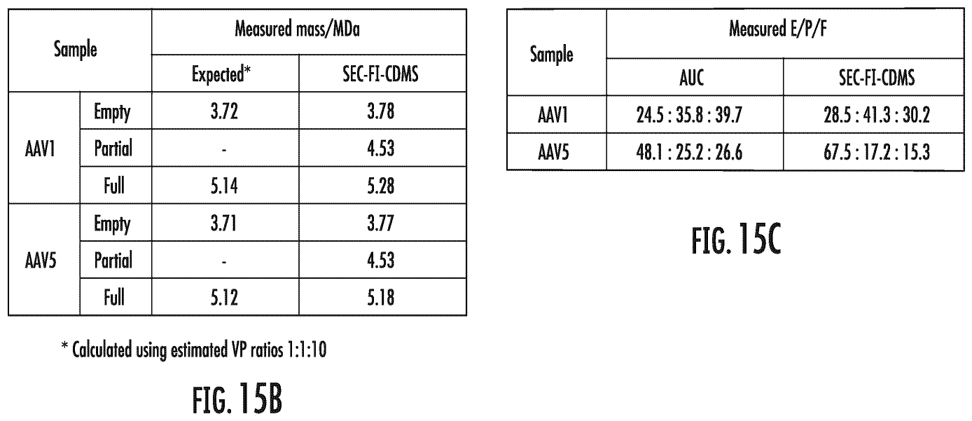

The developed platform was further applied to analyze other AAV serotypes with a higher amount of partial capsid contamination, for example AAV1 and AAV5. Usually, the partial capsids cannot be easily distinguished in conventional native MS spectra. However, with CDMS, partial capsids could be more clearly identified, as shown in FIG. 15A. The masses of empty, partial, and full capsids measured by SEC-FI-CDMS aligned well with expected values, as shown in FIG. 15B, and with those obtained from an alternative CDMS method. The relative abundance of empty, partial, and full capsids showed a similar trend across three analytical methods: AUC, an alternative CDMS method, and the developed SEC-FI-CDMS platform, as shown in FIG. 15C. The discrepancies in abundance are likely due to different ages of the samples. It should be noted that the abundance data from CDMS are estimated using mass range, and careful peak fitting is necessary to calculate accurate abundance. The average masses of empty, partially-filled and full vectors of each serotype is shown in Table 2. Overall, the developed platform was capable of detecting and quantifying partial capsids, and may be applied to enhance quality and purity assessment of manufactured AAVs.

TABLE 2

Average measured mass/MDa

| | | |
|---|---|---|
| Sample | MDS* | STORI |

| | | | |
|---|---|---|---|
| AAV1 | E | 3.77 | 3.76 |
| | P | 4.53 | 4.50 |
| | F | 5.22 | 5.23 |
| AAV5 | E | 3.77 | 3.71 |
| | P | 4.49 | 4.48 |
| | F | 5.17 | 5.18 |

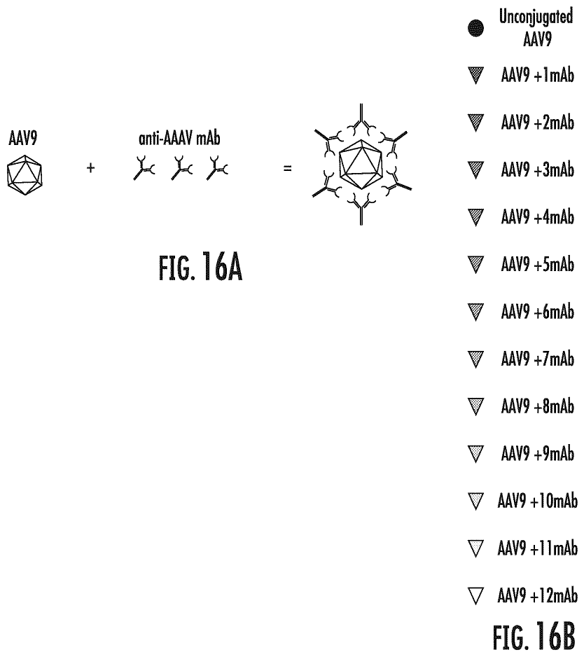

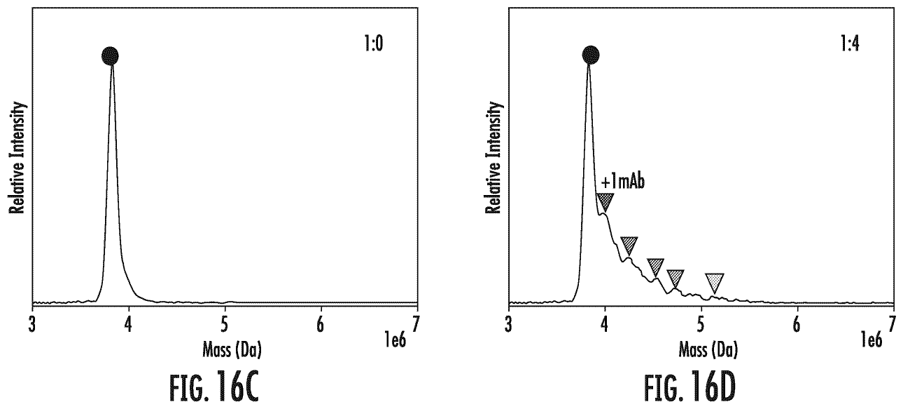

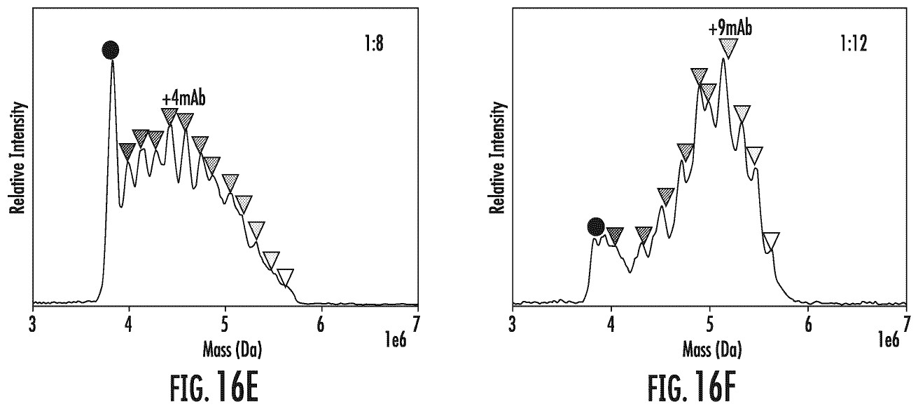

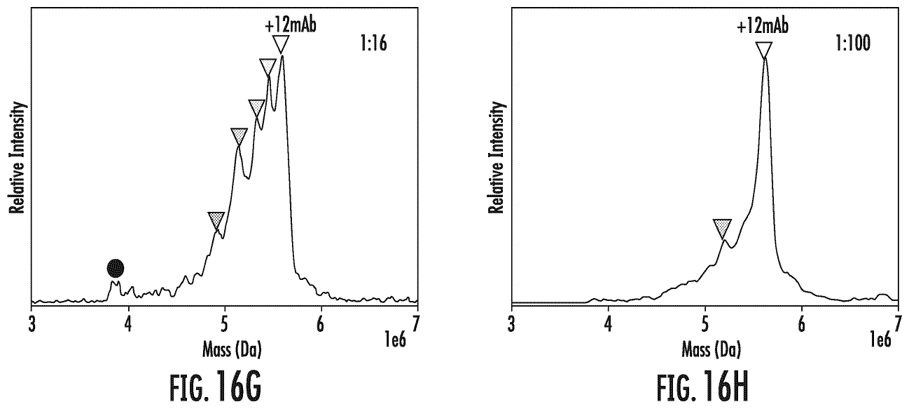

SEC-FI-CDMS was additionally shown to be capable of resolving variations of AAV-mAb conjugation as a function of increasing anti-AAV mAb, as illustrated in FIG. 16A. Anti-AAV mAb can bind to 5-fold axis of capsids with 12 theoretical binding sites. The degree of SpyC-mAb decoration of AAV9-SpyT with increasing ratios of SpyC-mAb to AAV9-SpyT as measured by the developed Orbitrap-based SEC-FI-CDMS method was assessed, as shown in FIGS. 16C-16H, see FIG. 16B for legend of FIGS. 16C-16H. The results from the developed SEC-FI-CDMS method were comparable to an alternative CDMS method. Compared to a naked AAV9 control, a mass shift to higher mass range was observed in the CDMS spectra of AAV conjugates by a stepwise addition of antibody mass.

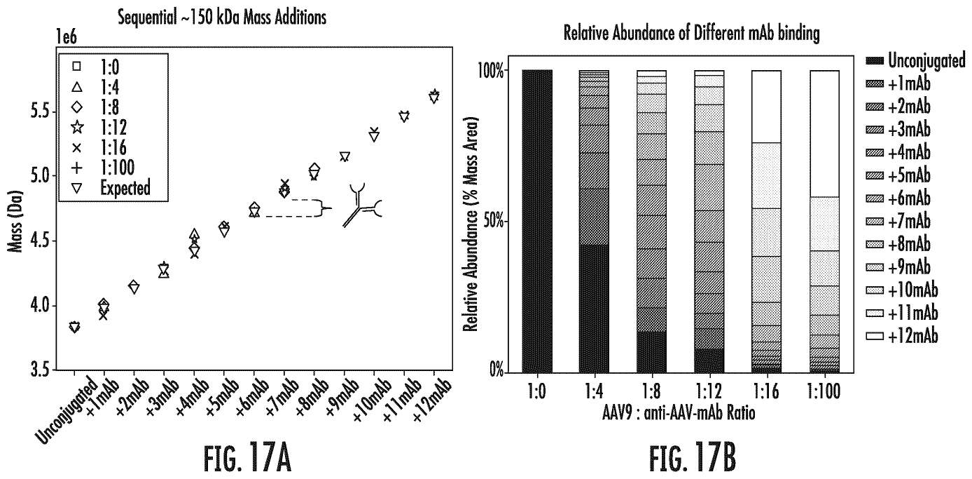

Up to 12× mAb binding was observed at the highest mAb concentration, and individual mAb addition can be well resolved, as shown in FIG. 17A. The distribution of mAb bound to AAV9 indicates a broad range of affinities, with one small component that resists binding a single antibody and another that binds over 12 antibodies under the same conditions, as shown in FIG. 17B. This approach allows efficient and robust measurements of the distribution of antibody decoration in AAV-antibody conjugates.

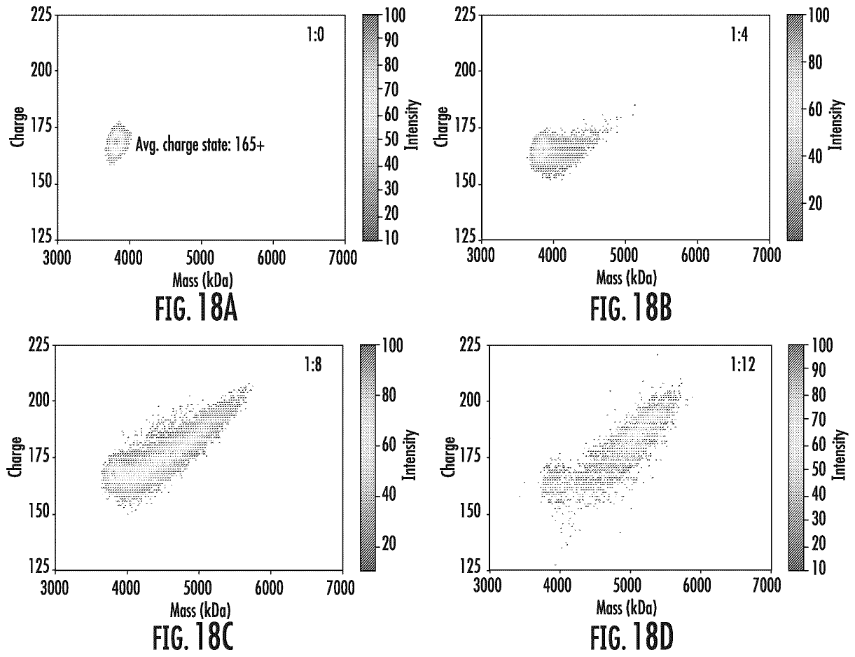

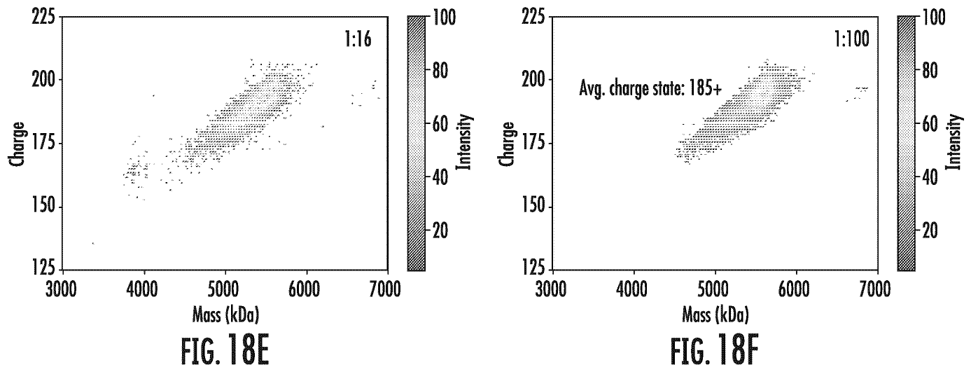

The SEC-FI-CDMS platform was further applied to reveal changes in AAV surface charge as a function of anti-AAV mAb binding. Two distributions of charge states were observed for AAV-antibody conjugate samples in the charge-mass heatmap, which correlated with naked AAVs and antibody-conjugated AAVs, respectively, as shown in FIGS. 18A-FIG. 18F. This result suggests a surface charge change upon antibody conjugation, demonstrating the value of measuring charge and mass simultaneously using the CDMS approach.

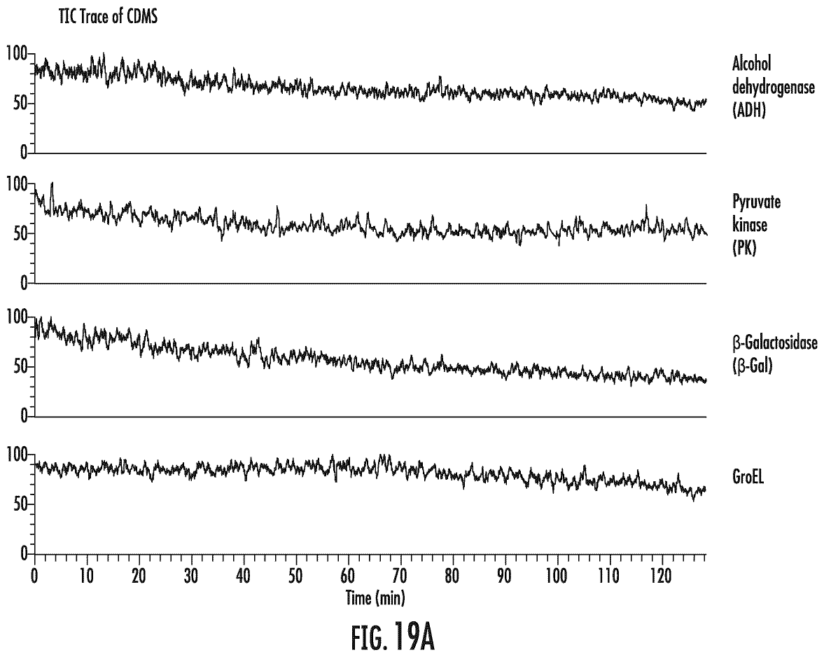

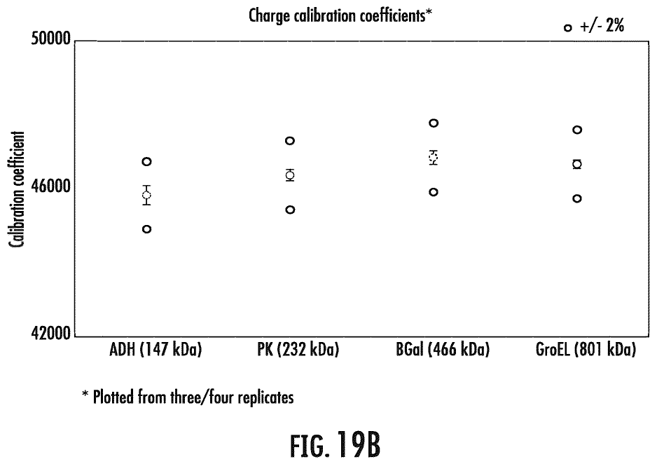

The developed SEC-FI-CDMS platform was also applied to establish CDMS charge calibration. SEC-FI enables the CDMS charge calibration function to be established using various protein standards. CDMS acquisition time of protein standards is over 120 minutes to ensure a reliable fitting of the charge calibration coefficient, requiring a long and stable injection time. Charge calibration coefficients across four protein standards using the SEC-FI-CDMS platform was within a 2% difference, as shown in FIG. 19A and FIG. 19B.

The SEC-FI platform set forth in the present disclosure streamlines the sample introduction process through a dual high-flow/low-flow setup. The high-flow configuration allows for fast online desalting and native MS screening, which can be applied for quick assessment of the full/empty ratio of AAV vectors. Full/empty ratios were determined by the relative abundance of capsids from online SEC-MS spectra, which were in good agreement with those calculated by AUC.

The low-flow configuration facilitates stable, sensitive, and robust sample infusion and long acquisition times for CDMS. In particular, the low-flow configuration enables Orbitrap-based CDMS analysis for approximately 30 minutes to several hours with minimal sample dilution. The masses of empty, partial, and full AAVs from three AAV serotype samples were accurately determined by SEC-FI-CDMS, which showed good agreement with predicted values.

The optimized SEC-FI-CDMS platform enabled efficient and robust mass analysis for AAVs. The masses and relative abundance of different AAV components determined using SEC-FI-CDMS are consistent with those from calculation or from orthogonal methods. SEC-FI-CDMS resolves and quantifies the distribution of antibody in AAV-mAb non-covalent conjugates, which could be too complex to resolve using other methods. The unique charge detection capability of CDMS also provides valuable insights into the surface charge of AAV-mAb conjugates.

### Example 5. Application of SEC-FI-CDMS for Duel AAV Analysis

Dual-vector AAVs offer a novel approach for delivering therapeutic transgenes that exceed the packaging capacity of AAV. This is achieved through complementary gene fragments that are independently packaged and recombine in vivo. Despite the promise of this approach, traditional characterization techniques lack the resolution to accurately quantify capsids in these complex mixtures. The characterization and quantification of dual vector AAVs is crucial for understanding vector reconstitution efficiencies but challenging due to the inherent heterogeneity of capsids and the complexity of transgenes. To address this challenge, a method utilizing a size-exclusion chromatography (SEC)-based online desalting coupling to native charge detection mass spectrometry (CDMS) was used to characterize dual vector AAVs samples. This method effectively resolved AAVs packaging different transgenes, allowing for the precise quantification of components within complex mixtures. Additionally, the structural integrity, genome release, disassembly, and aggregation of dual vector AAVs were evaluated by monitoring changes in their mass and charge distributions after thermal stress.

Native SEC was performed on an Thermo UltiMate 3000 UHPLC equipped with a Waters Acquity BEH200 SEC column. To enable CDMS analysis, a sample loop was added to trap the desalted analytes, and a nano spray was achieved at a low flow rate of 1 L/min using a secondary pump. A Thermo Q Exactive UHAMR equipped with a MnESI source and a Newomics multinozzle emitter was used for native MS and CDMS analysis. CDMS spectra were acquired using Direct Mass Technology (DMT) and processed using Proteinaceous STORIBoard. The dual-vector AAVs mixtures were analyzed using UniDec CD and quantified using Gaussian fitting in Origin Pro.

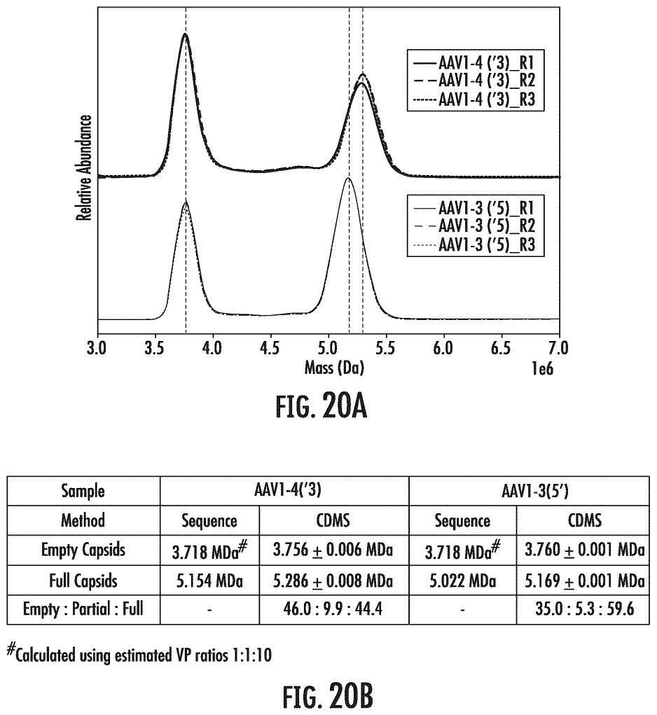

SEC-CDMS was used to resolve vectors comprising 3′ genomes or 5′ genomes based on payload mass. The observed mass difference between AAV1-4 (3′) and AAV1-3 (5′) is consistent with mass difference expected for the 3′ vs. 5′ payloads (~130 kDa) (FIGS. 20A and 20B). The percentage of full capsids determined by SEC-FI-CDMS corresponds closely with those from mass photometry (VPC), indicating that 50% and 65% of the capsids are full, respectively.

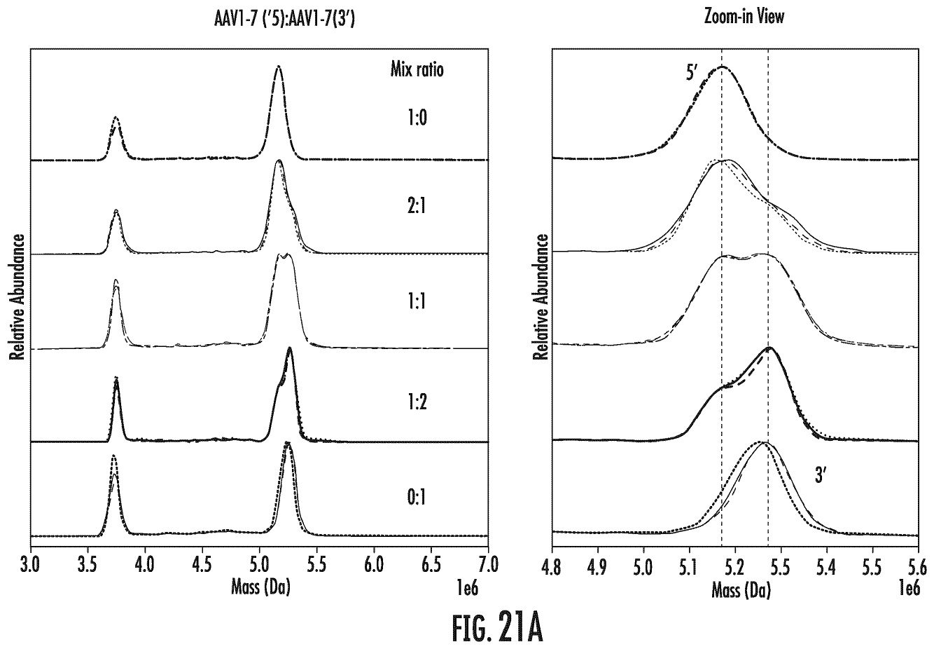

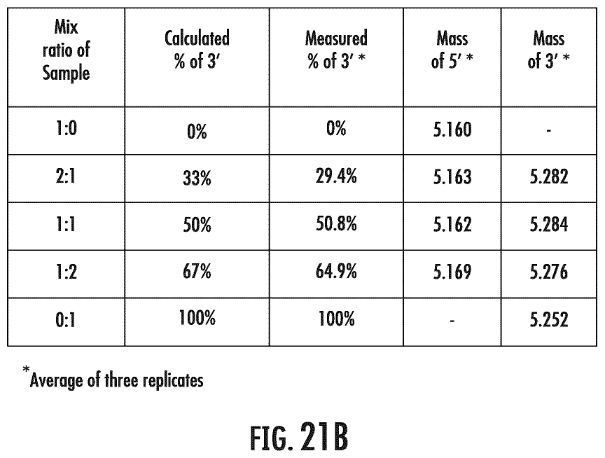

SEC-CDMS was used to resolve dual-vector AAVs in a mixture. Desalting SEC was directly coupled to CDMS using an extended sample loop for online analysis of AAV. This approach enables fast online buffer exchange and extended CDMS acquisition times for sensitive and robust mass analysis, and precise quantification of empty, partial, and full capsids. This technique was applied to characterize dual-vector AAVs by preparing a series of mixed samples with known genome titers of individual vectors, with expected ratios ranging from approximately 0:100 to 100:0. The individual vector components were well resolved from the mixtures based on a payload mass difference of ~130 kDa (FIGS. 21A and 21B). Quantification of each component using Gaussian peak fitting showed excellent agreement with expected values, with deviations within 4% (FIGS. 21A and 21B).

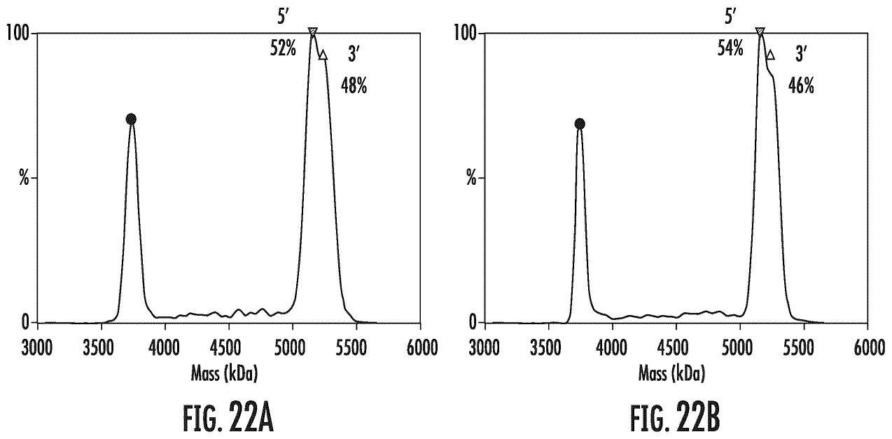

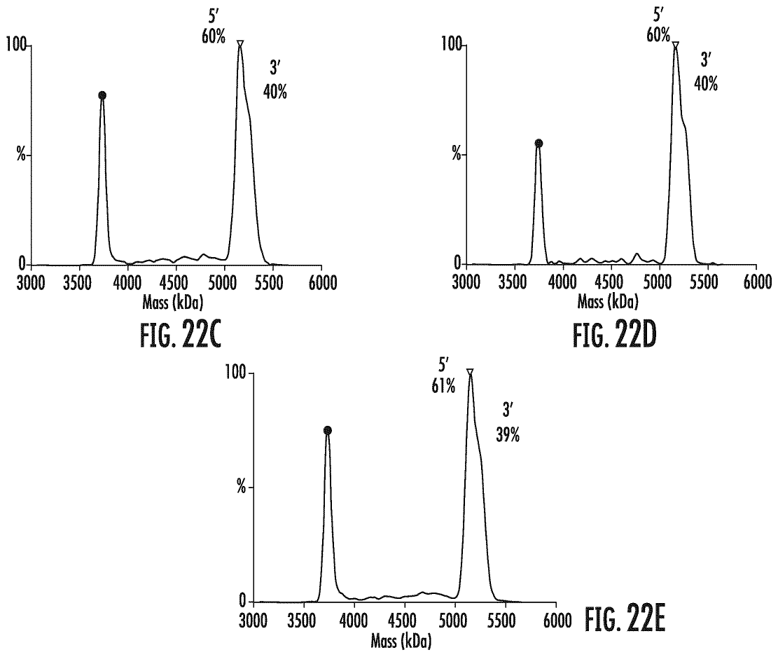

SEC-CDMS was used to resolve the difference of percentage of 5′/3′ full capsids at time 0, individual replicates. (FIGS. 22A-22E).

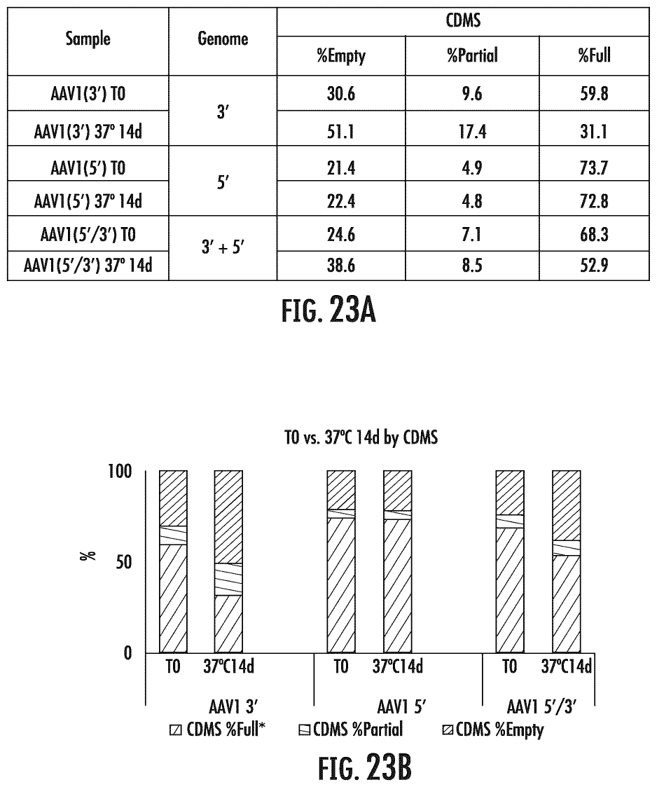

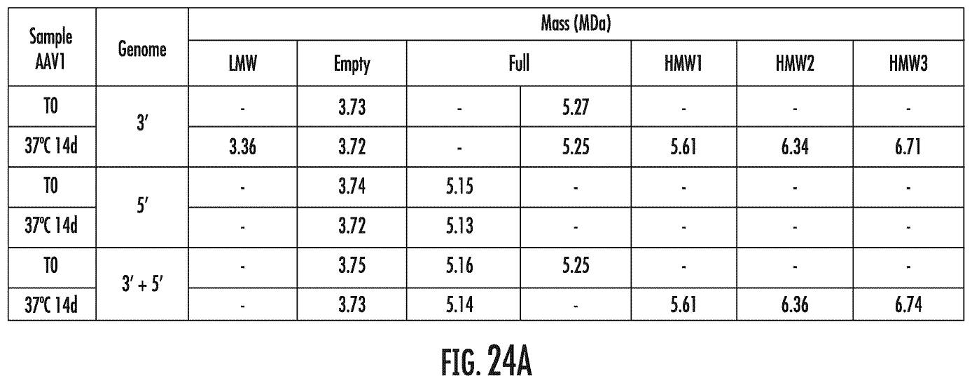

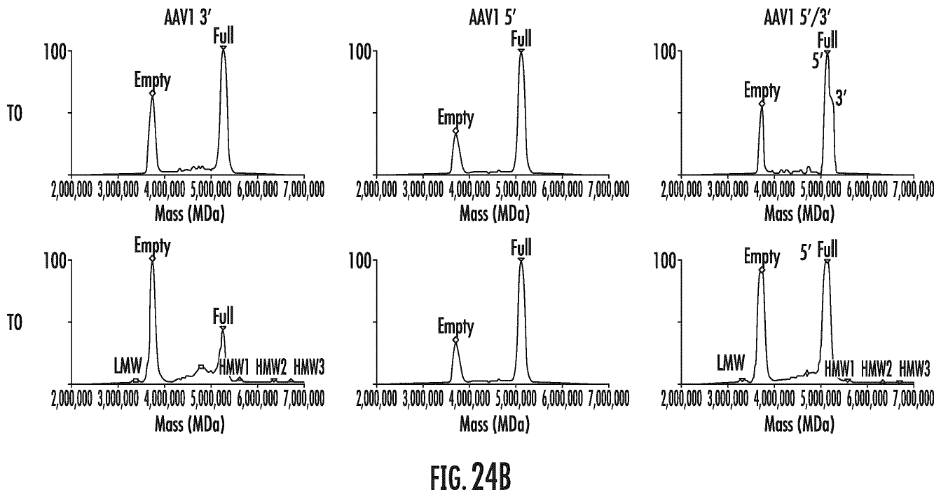

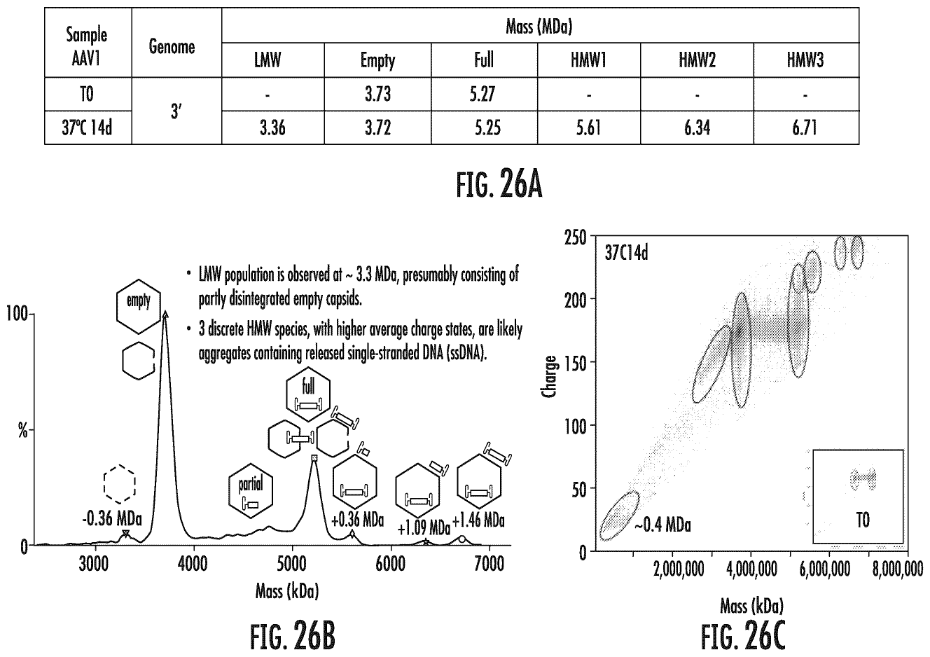

The method was further explored to investigate the thermal behavior of dual-vector AAVs after incubation. Upon thermal stress, individual AAV vectors exhibited unique behaviors. Vectors with shorter genomes demonstrated greater thermal stability, while those with longer genomes showed a depletion of full capsids and the emergence of novel size variants (FIGS. 23A and 23B). Dual-vector AAV mixtures displayed consistent behaviors of individual components. For AAVs with longer genomes, a population of LMW particles was detected, likely partially disintegrated empty capsids (FIGS. 24A-24B and 26A-26B). FIG. 26C depicts mass v. charge heat map of empty, partially filled, full, and heavy molecular weight (HMW) and low molecular weight (LMW) species of AAV1 comprising a 3′ template, according to an exemplary aspect.

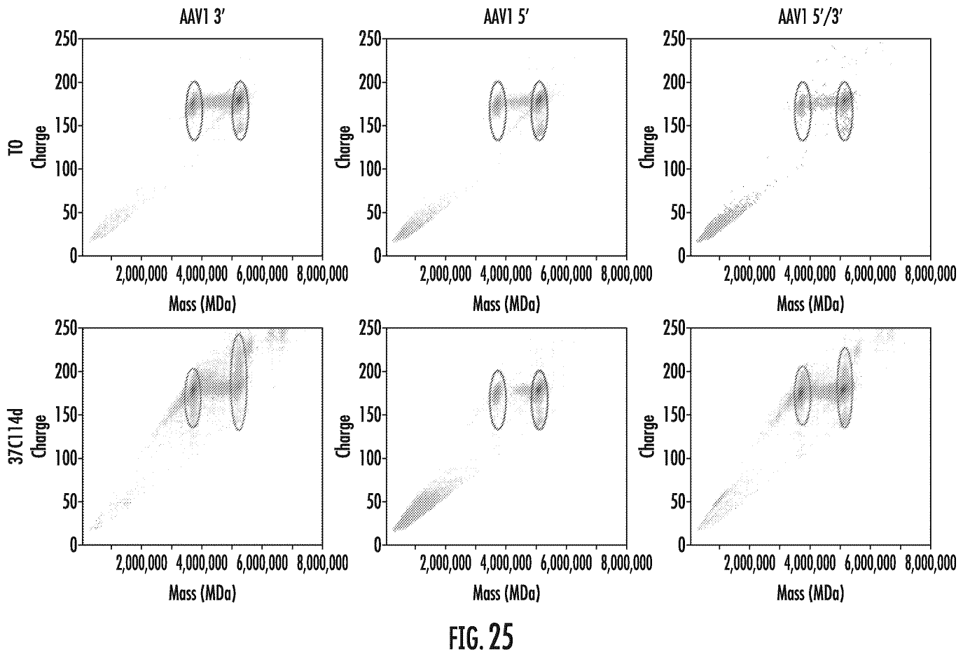

Mass versus Charge heat maps show stress stability of AAV vectors comprising a 5′ genome, 3′ genome, or both (FIG. 25). The empty and filled capsids of thermal stressed samples show broader charge distributions compared to the control samples. The HMW (heavy molecular weight) particles, presumably consist of aggregates with the released ssDNA, show higher average charge states than empty/full capsids.

Three HMW particles with higher average charge states than empty/full capsids were identified, likely aggregates containing released ssDNA. The decrease in full capsid percentage could be recovered with DNase treatment, providing insights into AAV aggregation and genome release.

Resolving challenges of dual-vector AAV characterization, quantification, and thermal stability analysis using novel SEC-based online desalting with native CDMS.

### Example 6. Charge Detection Mass Spectrometry (CDMS) and Native Digestion Strategies to Elucidate AAV Degradation Pathways

Thermal stress studies are commonly employed in formulation development to characterize the potential degradation pathways of AAV products under accelerated degradation conditions. When coupled with emerging techniques, such as mass photometry and charge detection mass spectrometry (CDMS), previous studies have revealed various capsid degradation pathways at global levels, including DNA extrusion and ejection, as well as capsid rupture and aggregation. However, such studies primarily use short exposures (e.g., 15-30 minutes) at high-temperatures (e.g., 65-80 °C) close to or above AAV melting temperatures (66.5-89.5 °C), where significant capsid structural changes occur. While effective for ranking thermostability, such conditions do not reflect the more moderate, prolonged stresses encountered in real-world processing, storage, or clinical-use settings. Moreover, degradation pathways at such high temperatures often differ fundamentally from those governing long-term stability at lower temperatures, limiting the predictive value of these accelerated degradation studies. As a result, the behavior of AAV capsids under mild yet extended thermal stress remains insufficiently characterized.

In addition, while the intricate relationship between DNA ejection and capsid degradation under thermal stress has been investigated, a detailed elucidation of the underlying mechanisms remains lacking. Some investigations have incorporated native DNase digestion with mass photometry, leading to the identification of DNA-extruded AAV species as an important degradation intermediate under thermal stress. However, the limited mass resolution of mass photometry restricts its ability to offer detailed structural insights into these intermediates. Other efforts have employed hydrogen deuterium exchange-mass spectrometry (HDX-MS) to demonstrate increased externalization of the VP1 N-terminal regions, concurrent with DNA ejection under thermal stress. In contrast, the present approach integrates CDMS and native digestion-assisted workflows to elucidate DNA ejection-induced AAV capsid degradation under thermal stress. Notably, the high-resolution mass measurements enabled by CDMS revealed multiple novel capsid degradation products resulting from DNA ejection. In addition to the previously described DNA-extrusion species, the present analysis also uncovered structurally compromised empty capsids that had lost a defined number of viral protein units as a result of DNA ejection. Furthermore, native enzymatic digestion targeting both DNA and protein components, followed by size exclusion chromatography, CDMS, and LC-MS analyses, confirmed the interplay between DNA ejection and VP1u/VP1/2 externalization. This comprehensive approach demonstrated the cooperative involvement of both AAV genome and viral proteins in AAV aggregation under thermal stress, revealing a DNA-viral protein-mediated aggregation pathway.

### Results

### DNA Ejection-Induced Capsid Degradation in Thermally Stressed AAV1 Studied by SEC-FLR UV and CDMS

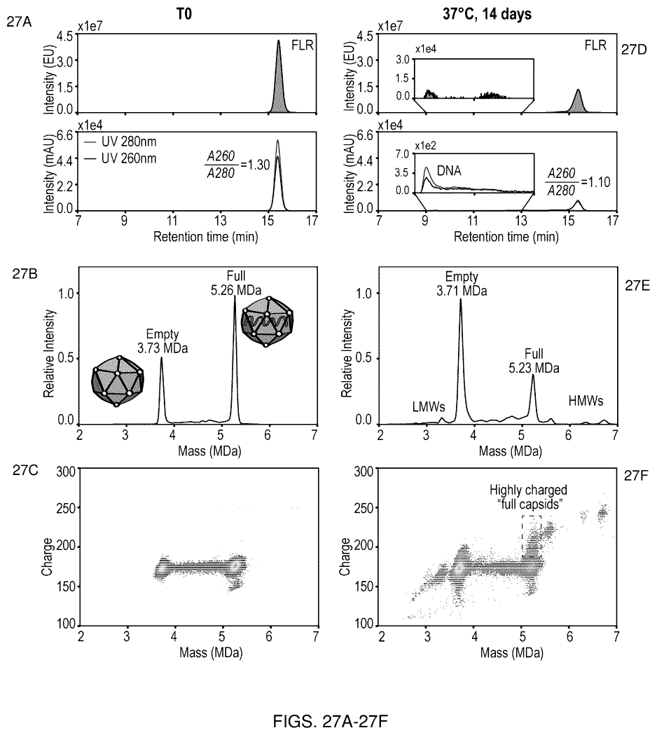

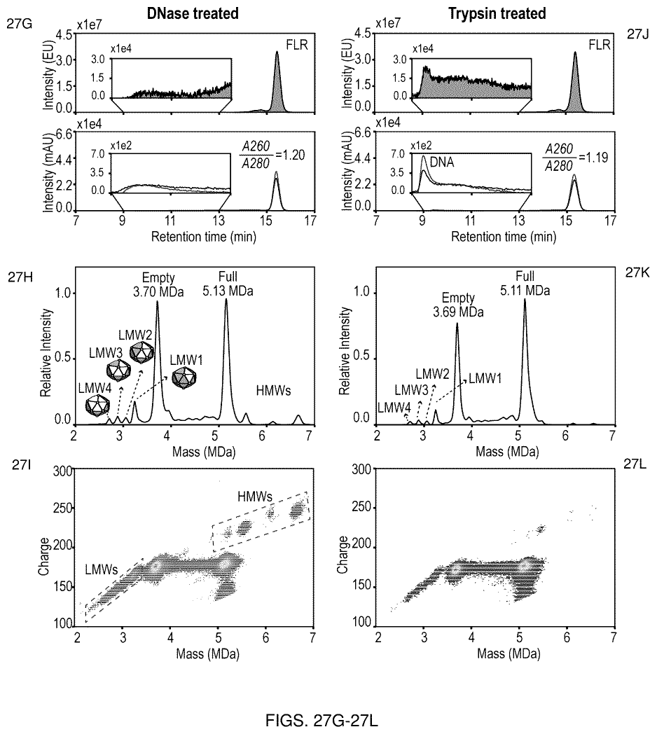

AAV1 capsids, encapsulating a ssDNA genome of ~4600 bases, were first analyzed at time zero (T0) and after a 14-day incubation at 37 °C using SEC-FLR/UV and CDMS (FIGS. 27A-27L). At T0, SEC-FLR/UV analysis revealed a predominant FLR peak corresponding to monomeric AAV1 particles, with an A260/A280 UV peak area ratio of 1.30 (FIG. 27A), indicating a mixture of both full and empty capsids. CDMS measurements provided further compositional resolution, revealing that approximately 58.4% of the capsids were genome-containing, with a molecular weight distribution centered at 5.26 MDa (FIG. 27B). Empty capsids accounted for 31.1% of the population and exhibited a molecular weight of 3.73 MDa. Both full and empty capsids displayed relatively narrow charge state distributions spanning 150+ to 190+(FIG. 27C). Following thermal stress, the monomeric AAV peak in the SEC-FLR chromatogram showed a substantial decrease (−54.3%) compared to T0 sample (FIG. 27D), indicating a significant loss of monomeric particles. Concurrently, the SEC-UV trace revealed the emergence of a high-molecular-weight (HMW) peak with an elevated A260/A280 ratio eluting between 9 and 10 minutes, consistent with the release of DNA from the capsids. This observation was accompanied by a decrease in the A260/A280 ratio of the main peak from 1.30 to 1.10, strongly supporting thermal stress-induced DNA ejection and presumable conversion of full capsids into empty ones. Subsequently, CDMS analysis corroborated these findings, showing a reduction in full capsids from 58.4% to 27.6% and a corresponding increase in empty capsids from 31.1% to 49.4% following thermal stress (FIG. 27E).

Importantly, CDMS also revealed the emergence of new low-molecular-weight (LMW) species ranging from 2.78 to 3.31 MDa, as well as HMW species spanning 5.60 to 6.72 MDa (FIG. 27E). These variants were not clearly detected by SEC-FLR/UV analysis due to limited resolution and sensitivity, highlighting the advantages of CDMS. The LMW species exhibited broad charge distributions from 100+ to 180+, with lower molecular weights corresponding to lower charges, which aligns with disrupted capsids or capsid fragments (FIG. 27F). Conversely, the HMW species showed significantly elevated charge distributions (from 200+ to 260+) than the main species, likely reflecting larger fused capsid assemblies. To determine the source of these size variants, an empty capsid-enriched AAV1 lot was subjected to the same thermal stress and subsequently examined by CDMS. Interestingly, both LMW and HMW species were absent in the empty capsid-enriched sample following thermal stress, indicating that their formation is specifically associated with full capsids. These observations suggest that DNA ejection not only converts full capsids into empty ones but also leads to the formation of structurally compromised capsids through the process, likely facilitated by the interaction between the genome and the viral proteins. In addition, the charge distribution of the remaining full capsids broadened significantly after thermal stress, with species exhibiting elevated charges up to 250+, as highlighted in the CDMS charge-to-mass heatmap (FIG. 27F). These highly charged “full capsids” likely represent intermediate DNA-ejection states, where DNA is partially extruded or remains tethered to the capsid surface, consistent with earlier reports (e.g., E. H. T. M., Ebberink et al., Molecular Therapy Methods & Clinical Development 2024, 32 (3)).

### Investigation of the Role of DNA Ejection and Viral Protein Externalization in Capsid Degradation Under Thermal Stress

To confirm the presence of DNA-extruded/tethered species and determine whether the ejected DNA was involved in newly formed size variants and aggregates, the thermally stressed AAV1 samples were treated with 10 μM DNase at 37 °C for 30 minutes to digest any externalized and solvent-accessible DNA. Following DNase treatment, the SEC-UV peak eluting at 9-10 minutes was substantially reduced (FIG. 27G), demonstrating that this peak was primarily composed of ejected DNA. Most strikingly, the recovery of the monomeric AAV particles was greatly improved, as shown by the increased main peak area in SEC-FLR analysis (FIG. 27G). This finding suggests that thermal stress likely causes DNA-mediated large aggregates that cannot be effectively recovered by SEC, whereas DNase treatment disrupts these aggregates, enabling increased SEC recovery of monomeric AAV particles. The increase in full capsid proportion following DNase treatment, as indicated by both the increased A260/A280 ratio (FIG. 27G) and CDMS data (FIG. 27H) further suggest that full capsids, compared to empty ones, contributed more significantly to aggregate formation in thermally stressed AAV1.

Furthermore, the LMW and HMW species remained after DNase treatment (FIG. 27H), indicating that they were mostly composed of viral proteins rather than DNA. Notably, the relative abundances of both LMW and HMW species rose following DNase treatment, suggesting they were likely involved in large aggregate formation. Owing to the improved signal after DNase treatment, CDMS analysis revealed that the LMW species likely correspond to structurally compromised empty capsids missing 6, 9, 12, or 15 VP units. Previous studies have shown that the interfaces between VP trimers represent relatively weak structural contacts within AAV assemblies (e.g., H.-J. Nam et al., Journal of Virology 2007, 81 (22), 12260-12271). Therefore, it is hypothesized that DNA ejection may extract VP units in multiples of three during the process, facilitated by the strong electrostatic interaction between the negatively charged DNA and the basic regions of the viral proteins. This process generates LMW capsid species that have lost 2-5 VP trimers, yielding distinct 45-, 48-, 51-, and 54-mer assemblies. These detailed insights highlight the benefits of high-resolution mass measurement from CDMS, particularly in comparison with other sizing techniques that offer comparatively lower resolution.

Lastly, the highly charged “full capsids”, as previously observed in CDMS charge-to-mass heatmap in stressed sample, largely disappeared following DNase treatment (FIG. 27I), confirming that these particles contained externalized DNA. This observation supports an intermediate DNA-ejection state under thermal stress, where DNA is either partially extruded or remains tethered to the capsid surface, leading to “full-like” capsids with elevated charge states.

The generation of VP-deficient LMW species from DNA-ejected full capsids, along with the formation of large DNA-mediated aggregates, both indicate that the interaction between viral protein and genome plays a crucial role during the DNA ejection process. Recent studies have demonstrated the cooperative action between DNA ejection and externalization of VP1 N-terminal regions (e.g., Y. Yamaguchi et al., Molecular Therapy Methods & Clinical Development 2025, 33 (2)). Because these regions contain clusters of basic residues, they are likely to interact with the genome and play an important role in DNA extrusion and ejection under thermal stress. To test this hypothesis, the thermally stressed AAV1 samples were further subjected to native trypsin digestion, targeting lysine (Lys) and arginine (Arg) residues from the externalized viral protein regions. Similar to DNase treatment, native trypsin digestion significantly increased the recovery of monomeric AAV particles by SEC-FLR analysis (FIG. 27J). Concomitantly, a substantial increase in the free-DNA peak (9-10 min) was observed in the SEC-UV trace (FIG. 27J). These results demonstrate that both ejected DNA and externalized viral proteins mediate the formation of large aggregates that are not effectively recovered by SEC but can be broken down by either native DNase or trypsin digestion. Consistently, the increased proportion of full capsids following native trypsin digestion was demonstrated by the increased A260/A280 ratio (FIG. 27J) and CDMS data (FIG. 27K), indicating their greater propensity in aggregation compared to the empty ones.

It is herein speculated that full capsids, with externalized basic protein regions (e.g., due to DNA extrusion), are particularly prone to aggregation by tethering with DNA released from other particles. Furthermore, the LMW species remained at notable levels after native trypsin digestion, suggesting they were relatively resistant to this treatment. In contrast, the abundance of HMW species decreased significantly, indicating these presumed fused capsid assemblies may be formed via the exposed protein regions that are susceptible to native trypsin cleavage. Additional structural characterization, such as TEM or cryo-EM, will be necessary to directly visualize these capsid size variants and provide further insights into their formation under thermal stress. Lastly, native trypsin digestion also effectively eliminated the highly charged “full capsid” species (FIG. 27L), supporting the hypothesis that thermal stress induces externalization of viral protein regions, which function as anchor points for DNA tethering on capsid surface. Cleaving the anchoring protein region via native trypsin digestion removes the tethered DNA, thereby eliminating the highly charged species. Altogether, this integrated CDMS and native digestion workflow revealed a cooperative process, where DNA ejection and externalization of viral protein regions occurred simultaneously under thermal stress, leading to a diverse class of degradation products.

AAV8 Capsid Degradation Under Thermal Stress Studied by SEC-FLR UV and CDMS with Native Digestion Strategies

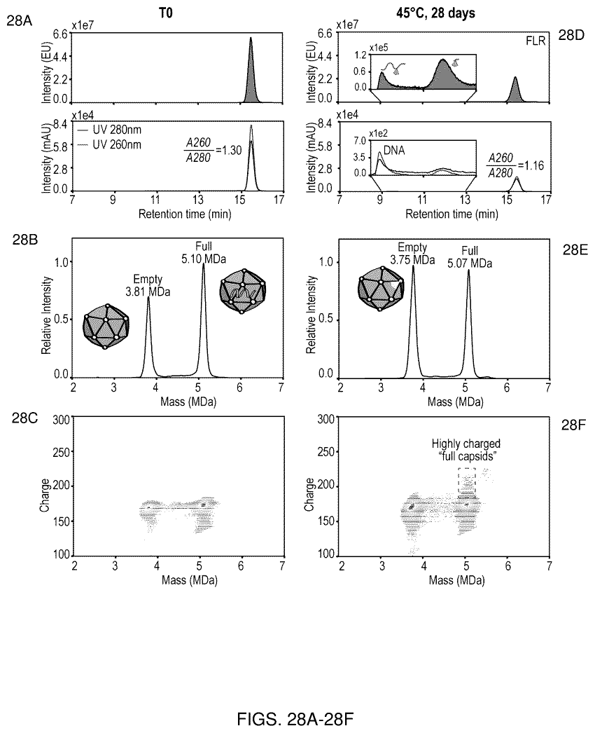

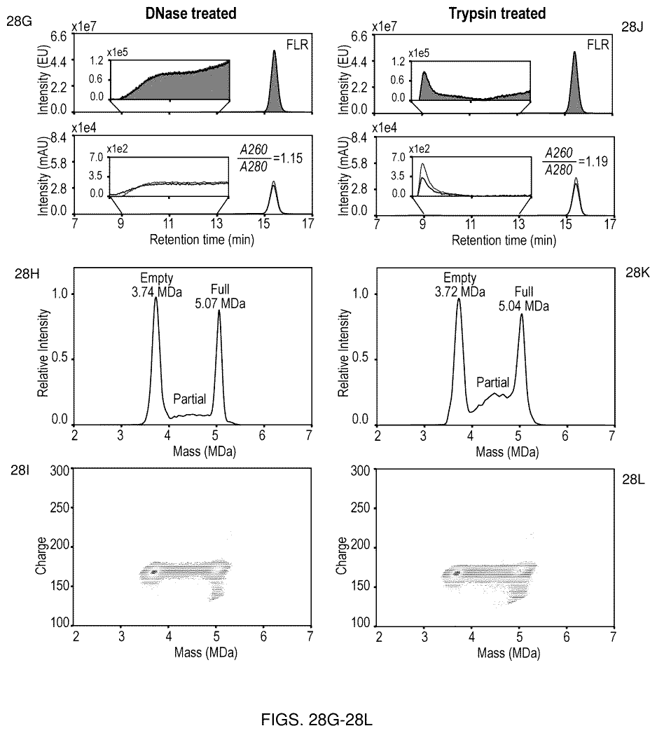

The same analytical strategies were used to study the thermal degradation behavior of another AAV8 sample, which contains a ~4,000 bases ssDNA genome. A harsher thermal-stress condition (45 °C for 28 days) was applied. Similar to AAV1, prolonged heat exposure caused a substantial loss of monomeric AAV particles, evidenced by a decrease in the SEC-FLR main peak area by 49.5% compared to T0 (FIGS. 28A, 2D). DNase or native trypsin treatments partially restored the monomer abundance, both increasing the SEC-FLR peak area to over 87.0% of T0 (FIGS. 28G, 2J). These changes in SEC monomer recovery indicate that thermal stress promotes large aggregate formation in AAV8, driven by interactions between ejected DNA and exposed viral protein regions, aligning well with the observations in AAV1. In addition, highly charged “full capsids” formed through DNA extrusion or tethering were also detected in stressed AAV8 (FIG. 28F) and were efficiently removed by either enzyme treatment (FIGS. 21, 2L).

A notable degradation feature of this AAV8 sample is the shift in full-capsid abundance following thermal stress and after native digestions. At T0, over 60% of AAV8 capsids were detected at a molecular weight centered at 5.10 MDa, consistent with genome-loaded particles (FIG. 28B). The percentage of full capsids decreased to 44.1% following thermal stress, indicative of DNA ejection, and dropped further to 35.8% and 40.0% after DNase and trypsin digestion, respectively. This observation indicates that, in contrast to the findings for thermally stressed AAV1, empty capsids played a more significant role in the formation of large aggregates. It is hypothesized that AAV8 may undergo a greater degree of DNA ejection due to the application of a more severe thermal stress condition (45 °C for 28 days in AAV8 versus 37 °C for 14 days in AAV1). The resulting empty capsids, with their basic protein regions externalized during DNA ejection, were highly susceptible to aggregation through interactions with free DNA released from other particles. Quantitative analysis of full capsids, achieved by multiplying the relative percentage of full capsids (as determined by CDMS) with the total SEC-FLR peak area, further support this hypothesis. Specifically, a net loss of 41.1% of full capsids was observed between T0 and the trypsin-treated stressed sample in AAV8, compared to a significantly smaller decrease of 10.1% in AAV1. Interestingly, following DNase or trypsin treatment, the percentage of partial capsids also increased from 3.1% to 9.7-20.7% (FIGS. 28H, 2K). It is hypothesized that prolonged exposure to a high temperature may also promote DNA hydrolysis (J. S. Plegaria et al., Molecular Therapy Methods & Clinical Development 2025, 33 (4)). The resulting DNA fragments, owing to their increased mobility, are more readily ejected under thermal stress, leading to the formation of partial capsids. These stress-generated partial capsids are highly prone to aggregation, due to the ejected DNA and externalized viral protein regions, making them difficult to detect in stressed samples unless enzymatic treatments are applied.

Another distinctive aspect of AAV8's thermal degradation behavior is the more subtle mass loss observed in empty capsids and the absence of apparent LMW or HMW species. At T0, empty AAV8 capsids had a mass of 3.81 MDa, which shifted to ~3.75 MDa after stress (FIG. 28E), indicating a loss of ~0.06 MDa. This decrease is larger than the 0.03 MDa mass loss observed in full capsids and considered significant relative to the measurement variability (±0.01 MDa). These results suggest that thermally stressed AAV8 may undergo a less destructive mode of DNA ejection and extract a single VP unit (~0.06-0.08 MDa) in the process, rather than generating apparent LMW species (e.g., losing VP oligomers) as observed in thermally stressed AAV1. Supporting this interpretation, the SEC analysis of stressed AAV8 revealed prominent HMW peaks with FLR signal (FIG. 28D), suggesting a significant presence of viral protein components. The first HMW peak (9-10 min, FIG. 28D), exhibiting both FLR signal and a greater A260/A280 ratio, disappeared after DNase treatment but persisted with native trypsin digestion (FIGS. 28G, 28J). This peak likely corresponds to the ejected DNA in association with few VP units. The second HMW peak (11-13 min, FIG. 28D), exhibiting prominent FLR signal but reduced A260/A280 ratio, largely diminished after either DNase or trypsin treatment (FIGS. 28G, 28J). This peak is likely attributable to much shorter DNA fragments in association with VP units, where the SEC retention times can be significantly altered by removing either the DNA or protein components. The distinct degradation profiles observed between AAV1 and AAV8 may be attributed to differences in serotype, genome size, and stress conditions. AAV1, with its larger genome, is more susceptible to physical rupture during DNA ejection, resulting in the formation of discrete LMW species. The lower temperature and shorter exposure also led to less pronounced full-to-empty and full-to-partial conversion. Conversely, AAV8, with relatively small genome, largely maintained the capsid structure after DNA ejection. However, the higher temperature and longer exposure led to more significant full-to-empty and full-to-partial conversion, due to accelerated DNA hydrolysis and DNA ejection. Systematic studies are still warranted to further explore the relationship between degradation profiles, AAV properties, and stress conditions. Nevertheless, these two examples highlighted distinct and diverse degradation products that may arise in AAV samples under thermal stress.

### Quantitation of Ejected/Extruded DNA in Thermally Stressed AAV Samples Using LC-MS-Based Nucleoside Analysis Following Differential Digestion Under Both Native and Denatured Conditions

The integrated CDMS and native DNase/trypsin digestion workflow indicates that DNA ejection/extrusion leads to the formation of diverse degradation products in AAV1 and AAV8 under thermal stress. To further assess the degree of DNA ejection/extrusion in thermally stressed AAV samples, an LC-MS-based method was developed for nucleoside analysis following complete DNA-to-nucleoside digestion under both native and denatured conditions. The digestion was achieved with Nucleoside Digestion Mix, an optimized enzyme blend that enables convenient and effective one-step generation of single nucleosides from DNA. Previous studies have shown that the encapsulated AAV genomes are highly resistant to DNase digestion under native conditions due to the protection from the viral capsid (Ebberink et al., Ibid.). As a result, the enzyme mixtures only digest the external DNA under native conditions, which may exist as unpackaged DNA impurities or be newly produced through DNA ejection/extrusion during thermal stress. Conversely, when the AAV samples are fully denatured, such as under high temperature and low pH conditions, the encapsulated genomic DNA is released and digested along with the external DNA. Subsequently, using LC-MS analysis, the percentage of externalized DNA relative to the total DNA in an AAV sample can be estimated by comparing the nucleoside signal obtained under both digestion conditions. As expected, four dominant nucleosides from DNA, known as dC, dA, dG, and dT were well separated and detected by LC-MS. A control sample, containing all the reagents and buffer, was also analyzed to establish the baseline level of DNA due to its pervasive presence. Among these nucleosides, dT exhibits the least interference from reagent control, accounting for only 0.2% signal relative to a denatured AAV digest sample. Therefore, the signal of dT, measured by the peak area from extracted ion chromatogram (EIC) in LC-MS analysis, was used as a proxy to estimate the abundance of total DNA (via denatured digestion) and externalized DNA (via native digestion).

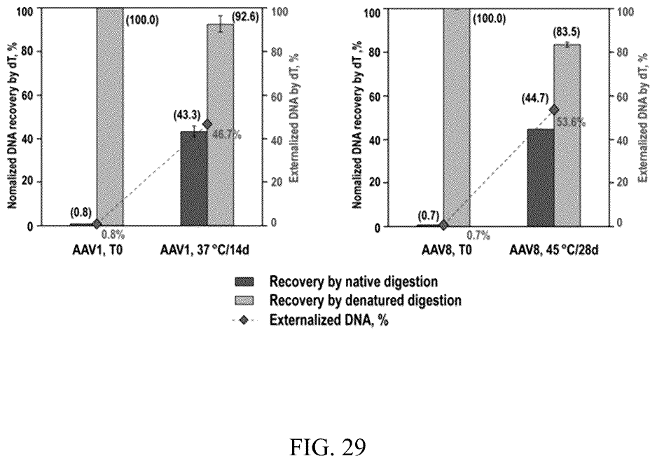

This method was then applied to both AAV1 and AAV8 samples at T0 and following thermal stress (FIG. 29). To assess the changes in overall DNA recovery due to thermal stress and under different digestion conditions, the dT signal from each sample was normalized against the value in T0, denatured digestion sample. For both AAV1 and AAV8 samples (shown in left and right graphs, respectively), the DNA recovery was much higher in the denatured digestion samples (FIG. 29, grey bars) than in the native digestion samples (FIG. 29, blue bars) regardless of thermal stress, matching the expectation that the encapsulated genomic DNA remains resistant to native digestion. Following thermal stress, the total DNA recovery, as measured by denatured digestion, was notably reduced to 92.6% for AAV1 and 83.5% for AAV8. This observation indicates that the externalized DNA, as a result of thermal stress, cannot be fully recovered even under the denatured digestion conditions, likely due to its propensity to form large aggregates. To estimate the degree of DNA externalization in each sample, the dT signal obtained by native digestion was further normalized against the value by denatured digestion (y-axis on the right, FIG. 29). At T0, both AAV samples exhibit less than 1% externalized DNA contents, indicating very low levels of free DNA contamination. These results are in line with what is expected from well-purified AAV products and agree well with literature findings using orthogonal, DNA dye-based techniques (e.g., K.-Y. Lai et al., Gene Therapy 2026; J. S. Bee et al., Journal of Pharmaceutical Sciences 2021, 110 (9), 3183-3187). Following thermal stress, the percentage of externalized DNA increases dramatically to 46.7% for AAV1 and to 53.6% for AAV8, demonstrating substantial DNA extrusion and ejection in both samples. The greater degree of DNA externalization and reduced total DNA recovery observed in AAV8 sample are also consistent with CDMS findings, reflecting the more intense thermal stress applied.

### Identification of Externalized Viral Protein Regions in Thermally Stressed AAV Samples Using Native Trypsin Digestion and LC-MS

To identify the externalized viral protein regions responsible for DNA tethering and large aggregate formation, a native trypsin digestion strategy was applied. This approach targets externalized viral protein regions that are rich in Lys and Arg residues. Several studies have shown that AAV capsids are highly resistant to trypsin digestion under native conditions, owing to its exceptional structural stability. Such features have previously been explored for improved host cell proteins (HCPs) detection using LC-MS-based techniques. To identify viral protein regions that are accessible to native trypsin digestion, peptide mapping analysis was performed on both AAV1 and AAV8 samples after incubating them with trypsin (enzyme-to-substrate ratio of 1/25) in pH 7.5 Tris buffer at 37 °C overnight. Tryptic peptides derived from the VP3 region were detected at minimal intensity in both AAV serotype samples. In contrast, peptides originating from the VP1u and VP1/2 regions were detected at levels more than 100-fold higher, even though VP1 and VP2 are present in much lower copy numbers relative to VP3. These results indicate that, independent of thermal stress, the VP3 region remains resistant to native trypsin digestion, reflecting its fundamental role in maintaining capsid structure. Conversely, the VP1u and VP1/2 regions exhibit markedly greater susceptibility to native trypsin digestion, seemingly contradicting the common notion that these regions are predominantly situated within the capsids. This observation suggests that, beyond their lack of compact structures, these regions may undergo dynamic externalization, aligning with findings from previous reports (e.g., X. Ye et al., Protein Science 2024, 33 (7), e5074).

Although peptide mapping analysis provides site-specific cleavage information, quantitatively assessing the digestion susceptibility of these regions remains challenging. This difficulty arises from the uneven distribution of Lys and Arg residues, as well as the MS response discrepancy among peptides. More critically, peptide mapping analysis struggles to distinguish the digestion susceptibility between VP1 and VP2 due to their shared VP1/2 region. To overcome these limitations, next experiments employed a denaturing SEC-MS method to quantify changes in the relative abundance of intact VP1, VP2, and VP3 following native trypsin digestion. In undigested samples, dSEC provides clear separation of all three VPs. The relative abundance of VP1, VP2 and VP3 is 9.0%, 9.0%, and 82.0% for unstressed AAV1 sample, and 8.3%, 10.9, and 80.8% for unstressed AAV8 sample, respectively. Interestingly, thermally stressed samples showed a newly formed HMW peak corresponding to VP3 dimer, as well as notable decreases in relative abundance of VP1 for both AAV1 (from 9.0% to 6.3%) and AAV8 (from 8.3% to 6.2%). The preferential loss of VP1 aligns with CDMS observations, suggesting that a fraction of VP1 proteins were extracted during DNA ejection under thermal stress due to favorable genome interaction, making them unrecoverable even under the denaturing conditions.

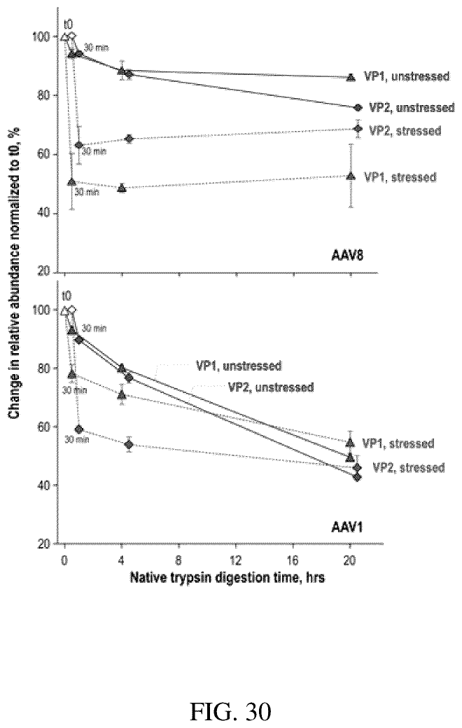

To further assess the digestion susceptibility of individual VPs before and after thermal stress, a native digestion time-course experiment (t=0, 0.5, 4, and 20 h) was performed for both AAV1 and AAV8, followed by dSEC-FLR/MS analysis. In this workflow, tryptic cleavages within VP1u or VP1/2 regions from VP1 will result in reduced abundance of VP1, whereas tryptic cleavages within VP1/2 region from VP2 will reduce VP2 abundance. Therefore, the digestion susceptibility of VP1 and VP2 can be readily distinguished. Consistent with peptide mapping analysis, both AAV1 and AAV8 samples, regardless of thermal stress, showed preferential loss of VP1 and VP2 abundances following native trypsin digestion. To better visualize the trend and account for run-to-run signal variation, the changes in relative abundance of VP1 and VP2, normalized to t0 values, were plotted against the digestion time (FIG. 30). For the unstressed AAV8 sample, the abundances of VP1 and VP2 decreased gradually over the first four hours, then remained relatively stable overnight (FIG. 30). In contrast, the abundances of VP1 and VP2 in unstressed AAV1 exhibited a continuous decline throughout the entire digestion time course. These distinct digestion trends may reflect the inherent differences between the two AAV samples, particularly with regard to their dynamics of VP1 and VP2 externalization. Following thermal stress, both AAV1 and AAV8 samples showed a rapid loss of VP1 and VP2 abundance within the first 30 minutes of native digestion (FIG. 30), which supports the idea that thermal stress induces increased externalization of VP1u and VP1/2 regions and renders them readily accessible to native trypsin digestion. Notably, thermally stressed AAV8 exhibited a faster digestion rate of VP1 compared to VP2. This observation may be explained by the fact that all the trypsin cleavage sites found in VP2 are also present in VP1. It may also suggest that VP1 externalization is more closely associated with DNA ejection, which is more pronounced in AAV8 due to the harsher thermal stress. Interestingly, an opposite trend was observed in stressed AAV1, where VP2 was more rapidly digested than VP1. It is hypothesized that the externalization of VP2 N-terminal region (i.e., VP1/2) may be more prominent than VP1 under milder thermal stress conditions, less dependent of DNA ejection. Owing to its small size, lack of structure, and absence of apparent genome interaction, the VP1/2 region from VP2 may exhibit greater dynamics and mobility through the 5-fold symmetric pore, rendering it susceptible to native trypsin digestion. This also explains in both unstressed AAV1 and AAV8 samples, that VP2 showed greater susceptibility to native digestion than VP1, despite containing fewer tryptic cleavage sites (FIG. 30). Collectively, the integration of native digestion strategy with peptide mapping and dSEC-MS-based intact VP analysis establishes that thermal stress induces externalization of VP1u and VP1/2 regions from both VP1 and VP2. In particular, the externalization of VP1u likely occurs cooperatively with DNA ejection, with the externalized VP1u functioning as the primary anchor points for subsequent interactions with ejected DNA, thereby driving the formation of both DNA-tethered intermediate specie and eventually large DNA-capsid aggregates.

### DISCUSSION

Thermal stressed-induced capsid degradation of AAVs is a multifaceted process involving chemical modifications, genome ejection, capsid disintegration, and aggregation. These pathways are influenced by factors including temperature, pH, ionic strength, serotype, and genome size, making it challenging to predict AAV behavior under stress. For example, the impact of genome size on capsid stability remains controversial. Some studies report that larger genomes destabilize the capsid and promote DNA release at lower temperatures, whereas others have shown that capsids packaging genomes near the wild-type size (~4.7 kb) exhibit maximal stability, with both shorter and longer genomes releasing their contents at temperatures below empty-capsid disassembly. These conflicting observations underscore the complex interplay between genome size, internal pressure, and DNA-capsid interactions. Similarly, the relationship between genome release and capsid disassembly is not fully resolved. Prior studies have proposed that heat induces N-terminal exposure of VP1 and VP2, enabling genome release while maintaining overall capsid integrity. Other reports, however, indicate that genome ejection destabilizes the particle and leads to capsid disassembly. These discrepancies highlight the need for mechanistic studies capable of resolving intermediate species and distinguishing between coexisting degradation pathways.

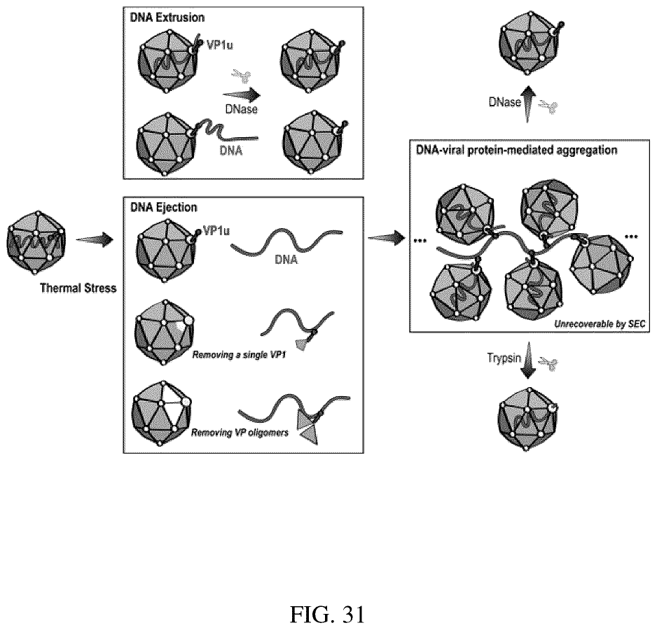

The present analysis provides direct mechanistic insight into these unresolved questions through an integrated analytical framework. The present work identified DNA-extrusion intermediates that retain the mass of full capsids but exhibit elevated charge states in CDMS, and these were further validated by native DNase digestion (FIG. 31). Such intermediates support a stepwise, rather than abrupt, ejection mechanism. Their detection also highlights limitations in conventional DNA assays, such as ddPCR and fluorescent dye-based methods, which can misinterpret or under-represent partially extruded genomes. The combination of differential DNA digestion and LC-MS-based nucleoside analysis provides an orthogonal approach of quantifying externalized DNA.

A key mechanistic insight from this work is the cooperative action between DNA ejection and viral-protein externalization, which leads to distinct size variants and large aggregates (FIG. 31). By assessing the susceptibility to native trypsin digestion, the present work was able to rapidly localize the externalization of specific viral protein regions. Specifically, both VP1u and VP1/2 regions exhibit increased levels of externalization upon thermal stress, likely due to their favorable interactions with the genome. When DNA begins to emerge from the capsid, these interactions can pull the N-terminal domains outward, exposing protein segments that are otherwise buried in the native particle. These exposed regions, in turn, function as anchors that bind ejected or extruded DNA, driving the formation of large DNA-protein aggregates. These aggregates, due to their large size and complex composition, are not recoverable by SEC, leading to substantial loss of monomeric AAV particles. Importantly, it was herein found that these aggregates are largely reversible by either native DNase or native trypsin digestions. DNase digestion removes DNA tethers, and trypsin trims externalized protein regions, both restoring monomeric AAV recovery (FIG. 31). These new findings confirm that DNA-viral protein-mediated interactions, rather than irreversible structural collapse, dominate aggregation under the studied conditions. The formation of large DNA-protein aggregates has meaningful implications for product safety and efficacy. Aggregates are known to increase immunogenicity risk in protein therapeutics, and similar concerns apply to gene therapy vectors.

Interestingly, it was herein found that DNA ejection does not just yield intact empty capsids; instead, it can produce structurally compromised but distinct assemblies whose nature depends on serotype, genome size, and stress severity. For AAV8, which contains a smaller genome and experienced more extensive thermal stress, DNA ejection appears to displace only a single VP1 unit. In contrast, AAV1—with its larger genome and milder stress-released larger VP oligomers during the DNA ejection process (FIG. 31). These observations suggest that internal pressure, serotype-specific capsid architecture, stress temperature, and duration collectively determine how the particle responds to genome ejection.

### Materials and Methods

### AAV Samples and Native Enzyme Digestion for CDMS

The AAV1 and AAV8 capsids, including both T0 and thermal stressed materials, were produced and prepared. Native DNase treatment was initiated by adding 3 μL of 50 mM magnesium acetate and 3.25 μL water to 20 μL AAV (3.0E13 vg/mL) samples, followed by the addition 3.75 μL of 80 μM of DNase I to obtain final AAV and DNase concentrations of 2.OE13 vg/mL and 10 μM, respectively. For native Trypsin treatment, 1.2 μL of 0.1 mg/mL Trypsin was added at an estimated enzyme-to-substrate ratio (E/S) of 1:25, along with 3 μL of 500 mM Tris-HCl at pH 8 and 5.8 μL water. The enzyme/AAV mixtures were incubated for 30 minutes at 37 °C in a thermomixer.

### Native SEC-FLR UV Analysis

Native SEC-FLR/UV analysis was conducted using an ultrahigh-pressure liquid chromatography (UHPLC) system, equipped with a commercial SEC column (4.6×300 mm, 2.5 μm, 1000 Å). For the analysis, 5 μL of each AAV sample was injected and separated at a flow rate of 0.200 mL/min, with 150 mM ammonium acetate as the mobile phase. The UHPLC instrument settings included a fluorescence detector with an excitation wavelength of 280 nm, an emission wavelength of 348 nm, and an acquisition rate of 10 Hz. UV absorbance wavelengths of 260 nm and 280 nm were measured at an acquisition rate of 2.0 Hz. Peak area was calculated using commercial software.

### Orbitrap-Based Charge Detection Mass Spectrometry

Native charge detection mass spectrometry (CDMS) analysis was performed using a previously developed SEC-based online buffer exchange platform coupled with Direct Mass Technology™ mode on a Thermo Scientific™ Q Exactive™ UHMR (C. Du et al., Journal of the American Society for Mass Spectrometry 2025, 36 (10), 2230-2238). All AAV samples were on-line buffer exchanged into 150 mM ammonium acetate using a commercial UHPLC equipped with a commercial SEC column (1.0 mm×50 mm, 1.7 μm, 200 Å). To facilitate CDMS analysis, a sample loop was incorporated into the setup to trap desalted analytes, which were subsequently infused at a low flow rate of 1 μL/min using a secondary nano pump. CDMS data for AAV samples were collected over a duration of 60 minutes using instrument settings that included a spray voltage of 3.0 kV, capillary temperature of 400 °C, S-lens RF level of 200, optimized ion transfer for “high m/z,” in-source trapping at −28 V desolvation voltage and −50 V source DC offset, 200K resolution, and 1000 ms injection times. Mass distributions were analyzed using commercial software with a charge range of 100-300, a mass range of 2,000,000-8,500,000, bin sizes of 30 for m/z, 100 Da for mass, and 1 for charge, and FWHM settings of 100-300 for m/z and 5-10 for charge.

### Nucleoside Digestion of AAV Samples

Native nucleoside digestion was performed by adding 25 μL water to 10 μL AAV (3.0E13 vg/mL) samples, followed by the addition of 2 μL Nucleoside Digestion Mix and 10 μL 10× nucleoside digestion mix reaction buffer. The samples were then incubated at 37 °C for 1 hr in the thermomixer prior to LC-MS analysis.

For denatured nucleoside digestion, 10 μL AAV (3.0E13 vg/mL) samples were first denatured by adding 0.2 μL 250 mM acetic acid, followed by incubation at 80 °C for 10 mins. Later, 25 μL water, 2 μL Nucleoside Digestion Mix and 10 μL 10× nucleoside digestion mix reaction buffer were added and the samples were incubated at 37 °C for 1 hr in the thermomixer prior to LC-MS analysis.

### Nucleoside Analysis by LC-MS/MS

The digested nucleosides from AAV were analyzed by LC-MS using a commercial Orbitrap mass spectrometer coupled to a commercial UPLC system. The nucleosides were separated on a Waters™ ACQUITY Premier HSS T3 column (2.1 mm×150 mm, 1.8 μm, 100 Å) at a flow rate of 0.30 mL/min and 30 °C using a 35 min gradient as: 0 min 0% B, 5 min 0% B, 25 min 40% B, 25.5 min 95% B, 30 min 95% B, 30.1 min 0% B, 35 min 0% B. Mobile phases A and B consisted of 0.1% (v/v) formic acid in water (and 0.1% (v/v) formic acid in acetonitrile, respectively.

The MS parameters were set as: spray voltage of 3.5 kV, capillary temperature of 320 °C, sheath gas flow of 40, auxiliary gas flow of 10, and an S-lens RF level of 50. MS spectra were acquired from m/z 200-1000 in positive mode with a resolution of 70,000. The nucleoside peak area from native and denatured digests was extracted using commercial software for relative quantification of the externalized DNA percentage of AAV samples.

### Tryptic Digestion of AAV Samples

Native tryptic digestion (ND) was performed using ~5 μg of AAV capsids diluted to 12 μL in 100 mM Tris-HCl (pH 8.0). Trypsin was added at a 1:25 (w/w) enzyme-to-substrate ratio, and samples were incubated at 37 °C on a thermomixer for the designated time course (t=0, 0.5, 4 and 20 h). Reactions were quenched by adding 1.5 μL of 10% TFA, and samples were stored at 4 °C prior to dSEC-MS analysis.

### Peptide Mapping Analysis by LC-MS/MS

AAV capsid digests were analyzed by LC-MS/MS using a Thermo Scientific™ Orbitrap Exploris 480 mass spectrometer coupled to a Thermo Scientific™ Vanquish Flex UHPLC system. Peptides were separated on a Waters™ Acquity Premier Peptide CSH C18 column (2.1×150 mm, 1.7 μm, 130 Å,) at a flow rate of 0.25 mL/min and 40 °C using a linear gradient from 0.1% to 32% mobile phase B over 75 min, followed by an increase to 45% B over 15 min. Mobile phases A and B consisted of 0.1% (v/v) formic acid in water and 0.1% (v/v) formic acid in acetonitrile, respectively.

The mass spectrometer was operated with a spray voltage of 3.5 kV, capillary temperature of 320 °C, sheath gas flow of 35, auxiliary gas flow of 10, and an S-lens RF level of 50. MS1 spectra were acquired from m/z 300-2000 at a resolution of 120,000, and the top 15 precursors were selected for MS/MS analysis using HCD at a normalized collision energy of 30% and a resolution of 15,000. Raw data were searched using Byonic (v5.8.6, Protein Metrics), and peptide peak area from native and denaturing digests was extracted by appropriate software for relative comparison.

### Denaturing SEC-FLR/MS Analysis

Denaturing SEC-FLR/MS (dSEC-FLR/MS) analysis was performed using a previously established method (T. N. Tiambeng et al., Journal of Pharmaceutical and Biomedical Analysis 2025, 253, 116524). A total of 4 μL of either native digested or untreated AAV samples was injected onto a Waters™ GTxResolve™ Premier BEH SEC column (4.6×300 mm, 2.5 μm, 450 Å) installed on a Thermo Scientific™ Vanquish Horizon UHPLC system. The UHPLC system was coupled to a Thermo Scientific™ Orbitrap Exploris 480 mass spectrometer equipped with a Microflow-Nanospray Electrospray Ionization (MnESI) source and an 8-nozzle, 10 μm ID Microfabricated Monolithic Multinozzle (M3) emitter. The flow from the column was split at a 200:1 ratio between the mass spectrometer and the fluorescence detector.

VP1-3 separation was achieved under isocratic conditions using a mobile phase of 30.0% acetonitrile, 1.0% formic acid, and 5 mM ammonium formate at a flow rate of 0.125 mL/min. Samples were maintained at 60 °C on-column during analysis. Fluorescence detection was performed with an excitation wavelength of 280 nm and an emission wavelength of 348 nm at an acquisition rate of 10 Hz. The mass spectrometer was operated in low-pressure mode with parameters aligned to previously reported settings (T. N. Tiambeng et al., Ibid.). MS data were used for VP identity confirmation, while fluorescence peak areas were used for quantitative analysis of VP abundance using commercial software.

While there have been shown and described what are at present considered the preferred embodiments of the invention, those skilled in the art may make various changes and modifications which remain within the scope of the invention defined by the appended claims.

What is claimed is:

1. A method of assessing an AAV sample that contains AAV capsids housing genetic material, the method comprising:
subjecting the AAV sample to enzymatic digestion; and
subjecting the digested sample to size exclusion chromatography (SEC) and/or charge-detection mass spectroscopy (CDMS) to detect AAV degradation products in the AAV sample.

2. The method of claim 1, wherein the enzymatic digestion is achieved by contacting the AAV sample with DNAse or protease (e.g., trypsin).

3. The method of claim 1, wherein the degradation products comprise empty capsids, capsids with partially externalized DNA, and/or aggregated AAV species.

4. The method of claim 3, wherein the empty capsids lack one or more VP regions.

5. The method of claim 1, wherein the method further comprises identifying and/or quantifying one or more degradation products in the AAV sample.

6. The method of claim 1, wherein the method further comprising quantifying the ratio of empty capsids to full capsids.

7. The method of claim 1, wherein the method further comprises quantifying empty capsids missing one or more VP regions.

8. The method of claim 1, wherein the method further comprises quantifying capsids with partially externalized DNA.

9. The method of claim 1, wherein the enzymatic digestion converts aggregated AAV species into monomeric AAV species, and wherein the method further comprises quantifying the amount of aggregated AAV species in the AAV sample by comparing SEC/CDMS results of the enzyme-digested AAV sample with SEC/CDMS results of the undigested AAV sample.

10. The method of claim 1, wherein the AAV sample, before enzyme digestion, has been subjected to a condition that induces degradation of AAV.

11. The method of claim 10, wherein the condition is a temperature above room temperature.

12. The method of claim 1, wherein the SEC is SEC-FLR/UV, which is SEC equipped with fluorescence and ultraviolet dual detection.

13. The method of claim 1, wherein said CDMS contains an electrospray ion source (EIS).

14. The method of claim 13, wherein the EIS is a microflow nanospray ion source.

15. The method of claim 1, wherein the SEC is integrated with the CDMS by direct flow injection of mobile phase from the SEC into the CDMS, wherein said mobile phase is subjected to a desalting mechanism before being injected into the CDMS.

16. The method of claim 15, wherein the desalting mechanism is achieved using a high-flow configuration that provides online desalting and native mass spectroscopy (MS) screening, wherein said high-flow configuration employs a flow rate of about 10 μL/min to about 30 μL/min through the SEC column to result in a desalted sample.

17. The method of claim 16, wherein the desalted sample is injected into an EIS of the CDMS.

18. The method of claim 1, wherein the SEC employs a mobile phase that contains a volatile buffer.

19. The method of claim 18, wherein said volatile buffer is ammonium acetate.

20. The method of claim 17, wherein a sample loop contains the desalted sample and connects the SEC with the EIS.

21. The method of claim 17, wherein the desalted sample is injected into the EIS using a low-flow configuration that employs a flow rate of about 0.5 μL/min to about 2 μL/min.
